(19) United States
(12) Patent Application Publication
(10) Pub. No.: US 2010/0144903 A1
(43) Pub. Date: Jun. 10, 2010

(54) METHODS OF DIAGNOSIS AND TREATMENT OF CROHN'S DISEASE

(75) Inventors: Kent D. Taylor, Ventura, CA (US); Jerome I. Rotter, Los Angeles, CA (US); Stephan R. Targan, Santa Monica, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/598,794

(22) PCT Filed: May 2, 2008
(86) PCT No.: PCT/US08/62531

§ 371 Date: Nov. 4, 2009

Related U.S. Application Data
(60) Provisional application No. 60/916,026, filed on May 4, 2007.

(51) Int. Cl. A61K 31/00; C12Q 1/68; A61P 1/00

(52) U.S. Cl. 514/789; 435/6

(57) ABSTRACT

In one embodiment, this invention provides methods of diagnosing and/or predicting susceptibility to Crohn's Disease by determining the presence or absence of risk haplotypes in IL23R, IL17A, IL17RA and/or IL12RB1 locus. In another embodiment, the invention provides methods of diagnosing and/or predicting susceptibility to Crohn's Disease in an individual by determining the presence or absence of risk haplotype at the IL12RB2 locus.

### GOVERNMENT RIGHTS

This invention was made with U.S. Government support on behalf of the National Institute of Diabetes and Digestive and Kidney Diseases (NIDDK) by NIDDK Grant P01 DK46763. The U.S. Government may have certain rights in this invention.

### FIELD OF THE INVENTION

The invention relates generally to the fields of inflammation and autoimmunity and autoimmune disease and, more specifically, to genetic methods for diagnosing and treating Crohn's Disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments provide methods of diagnosing susceptibility to Crohn's Disease in an individual, comprising determining the presence or absence of a first risk haplotype at the IL23R locus, the presence or absence of a second risk haplotype at the IL17A locus, the presence or absence of a third risk haplotype at the IL17RA locus, and the presence or absence of a fourth risk haplotype at the IL12RB1 locus, where the presence of four of the risk haplotypes present a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one of the risk haplotypes presents a greater susceptibility than the presence of none of the risk haplotypes. In another embodiment, the first risk haplotype at the IL23R locus comprises IL23R Block 2H1 and/or Block 3H1. In another embodiment, the first risk haplotype at the IL23R locus comprises a variant selected from the group consisting of SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID NO.: 6, SEQ. ID NO.: 7, SEQ. ID. NO.: 8, SEQ. ID. NO.: 9, SEQ. ID. NO.: 10 and SEQ. ID. NO.: 11. In another embodiment, the second risk haplotype at the IL17A comprises IL17A H2. In another embodiment, the second risk haplotype at the IL17A locus comprises a variant selected from the group consisting of SEQ. ID. NO.: 12, SEQ. ID. NO.: 13, SEQ. ID. NO.: 14 and SEQ. ID. NO.: 15. In another embodiment, the third risk haplotype at the IL17RA locus comprises IL17RA Block 2H4. In another embodiment, the third risk haplotype at the IL17RA locus comprises a variant selected from the group consisting of SEQ. ID. NO.: 16, SEQ. ID. NO.: 17, SEQ. ID. NO.: 18, SEQ. ID. NO.: 19, SEQ. ID. NO.: 20 and SEQ. ID. NO.: 21. In another embodiment, the fourth risk haplotype at the IL12RB1 locus comprises IL12RB1H1. In another embodiment, the fourth risk haplotype at the IL12RB1 locus comprises a variant selected from the group consisting of SEQ. ID. NO.: 22 and SEQ. ID. NO.: 23.

Other embodiments provide methods of treating Crohn's Disease, comprising determining the presence of one or more risk haplotypes at the IL12RB1 locus, and treating the Crohn's Disease. In another embodiment, one of said one or more risk haplotypes at the IL12RB1 locus comprises SEQ. ID. NO.: 22 and SEQ. ID. NO.: 23.

Other embodiments provide methods of determining a low probability relative to a healthy subject of developing Crohn's Disease, comprising determining the presence or absence of a protective haplotype at the IL12RB2 locus in the invidual, and diagnosing a low probability of developing Crohn's Disease, relative to a healthy subject, based upon the presence of the protective haplotype at the IL12RB2 locus. In other embodiments, the protective haplotype at the IL12RB2 locus comprises IL12RB2H4. In other embodiments, the protective haplotype at the IL12RB2 locus comprises SEQ. ID. NO.: 24, SEQ. ID. NO.: 25 and SEQ. ID. NO.: 26. In other embodiments, the individual is Ashkenazi Jewish.

Various other embodiments provide methods of diagnosing susceptibility to Crohn's Disease in an individual, comprising determining the presence or absence of one or more risk haplotypes at the IL12RB2 locus in the individual, and diagnosing susceptibility to Crohn's Disease based upon the presence of one or more risk haplotypes at the IL12RB2 locus. In other embodiments, one of said one or more risk haplotypes at the IL12RB2 locus is H3. In other embodiments, one of said one or more risk haplotypes at the IL12RB2 locus is H1. In other embodiments, the individual is Ashkenazi Jewish. In other embodiments, one of said one or more risk haplotypes at the IL12RB2 locus comprises SEQ. ID. NO.: 24, SEQ. ID. NO.: 25 and SEQ. ID. NO.: 26.

Other embodiments provide methods of treating Crohn's Disease, comprising determining the presence of one or more risk haplotypes at the IL12RB2 locus, and treating the Crohn's Disease. In other embodiments, one of said one or more risk haplotypes at the IL12RB2 locus comprises SEQ. ID. NO.: 24, SEQ. ID. NO.: 25 and SEQ. ID. NO.: 26.

Other features and advantages of the invention will become apparent from the following detailed description, taken in conjunction with the accompanying drawing, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (a)-(b) (prior art) depicts the IL23/IL17 pathway. Sketch of the basic protein components of the IL23/IL17 pathway, leading to the development of the Th17 cell and subsequent production of IL17, contrasted with the IL12 pathway, leading to the development of the Th1 cell. Redrawn after Weaver, 2007. (a) The IL12 pathway; (b) the IL23/IL17 pathway.

FIG. 2 odds ratio for Crohn's disease with number of risk haplotypes. Odds ratio for CD for the presence of 0, 1, 2, 3, or 4 risk haplotypes for IL23R, IL17A, IL17RA, and IL12RB1.

FIG. 3 (a)-(g) depicts HapMap Data for Control Population, and observed structure of genes from association studies. (a) Observed IL23R Structure; (b) Observed IL17A Structure; (c) Observed IL17RA Structure; (d) Observed IL12B Structure; (e) Observed IL12RB1 Structure; (f) Observed IL12A Structure; (g) Observed IL12RB2 Structure.

FIG. 4 depicts a table listing the association of IL17-IL23 pathway related haplotypes with Crohn's Disease. With the exception of IL23R H6 which contains the R381Q variant, haplotypes with frequency >5% are shown. Variants are reported as the nucleotide on the forward strand of the NCBI Genome Build 36 and dbSNP v 126, although as would be obvious to one of skill in the art, the results described herein apply also to the complementary reverse strand.

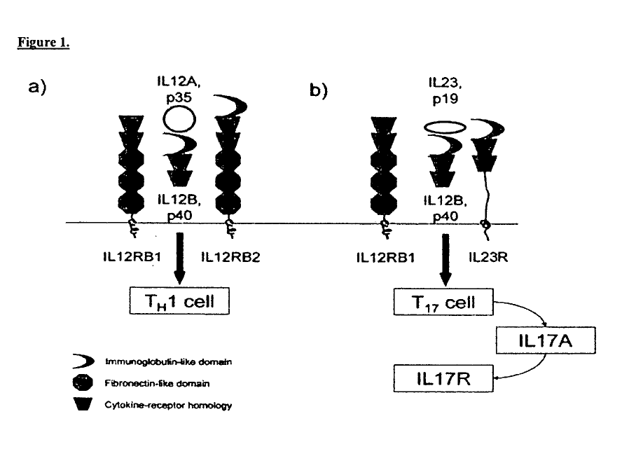

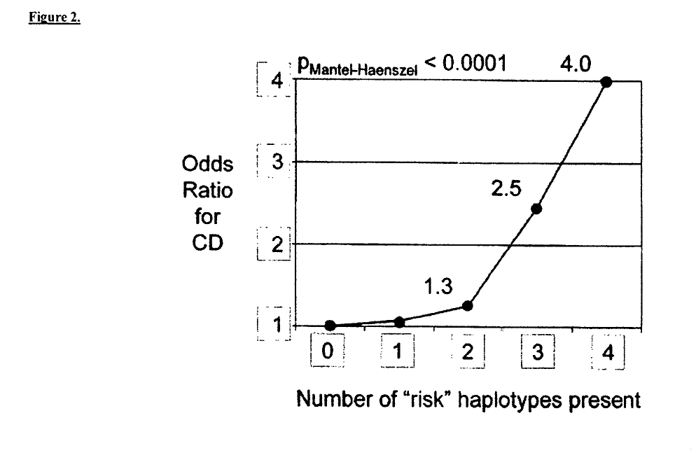

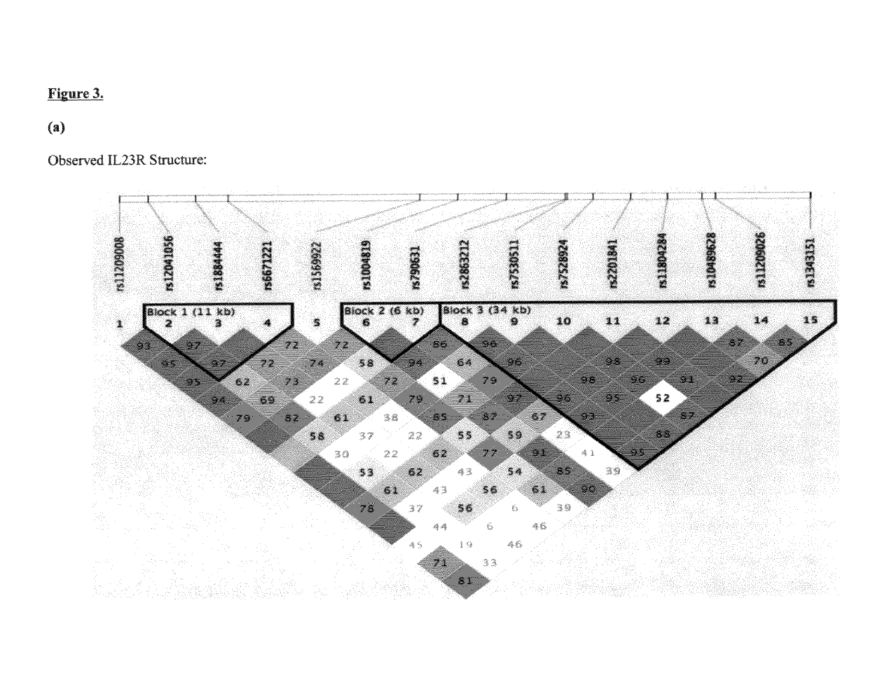

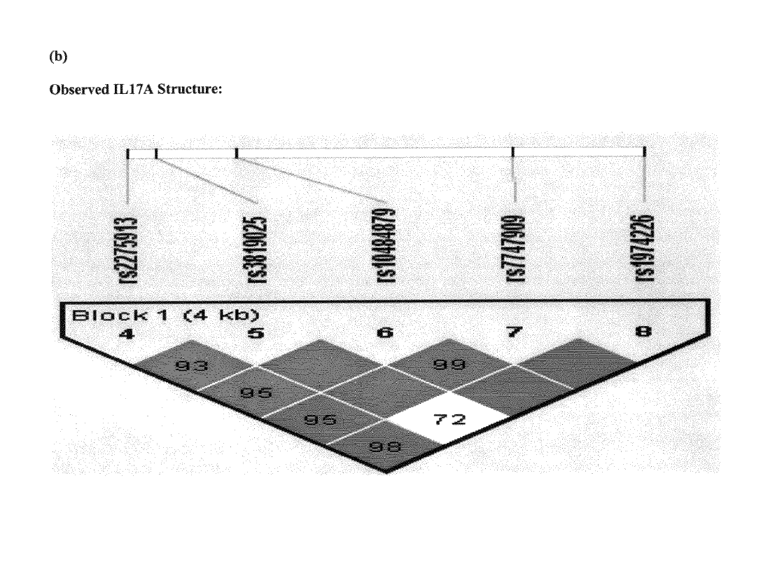

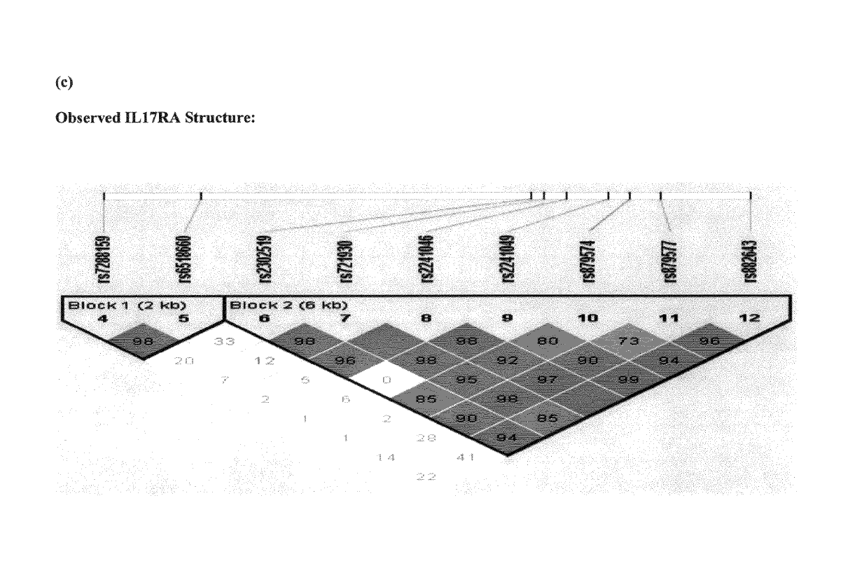

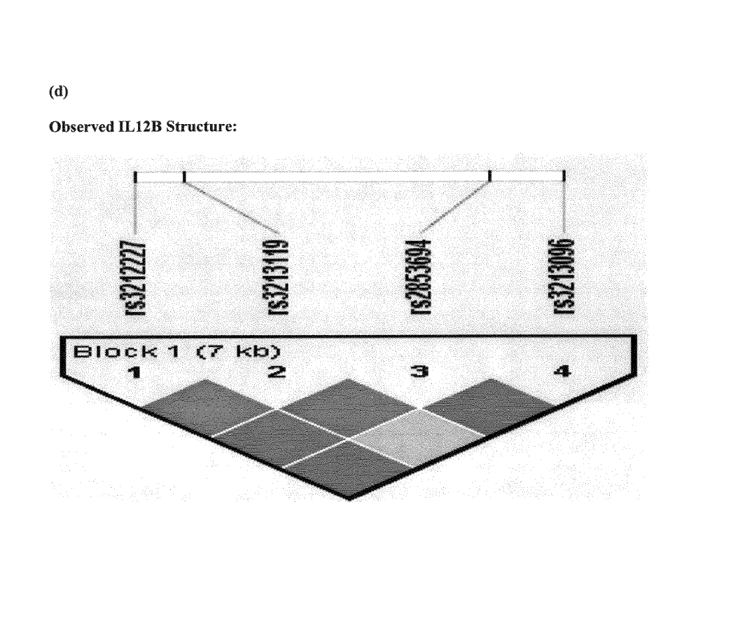

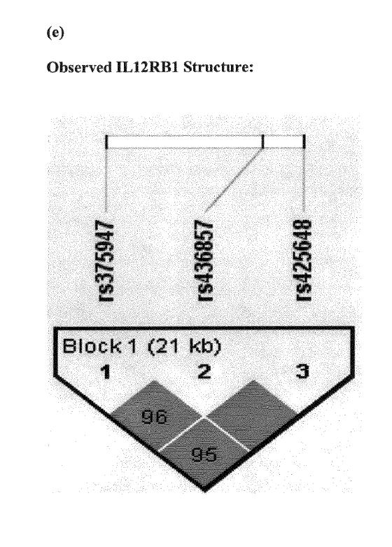

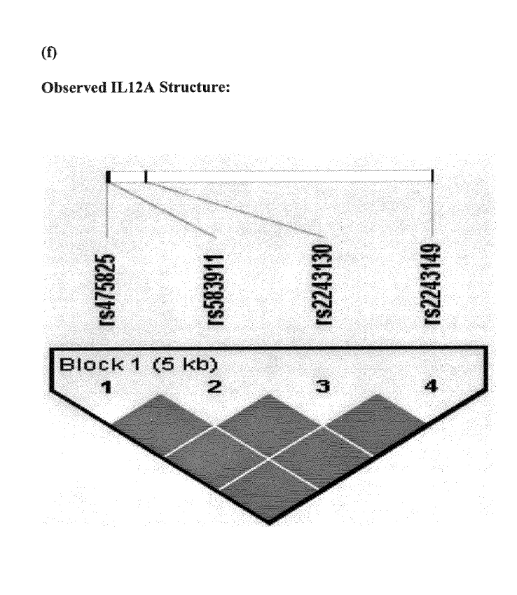

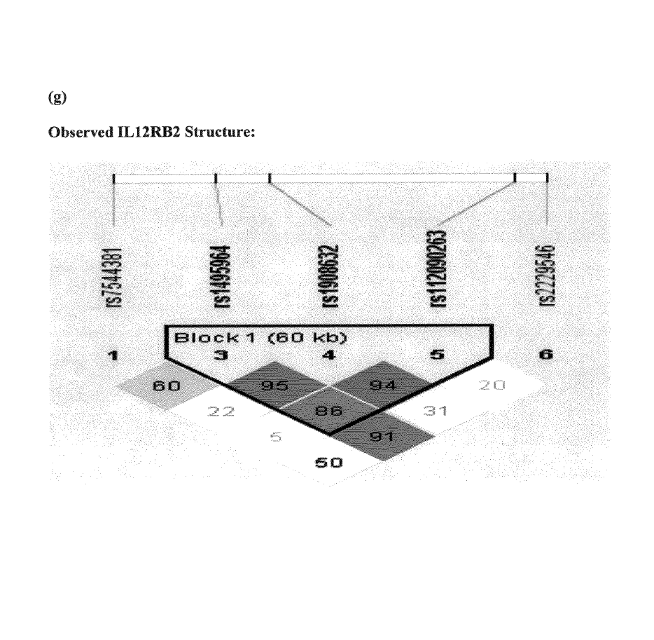

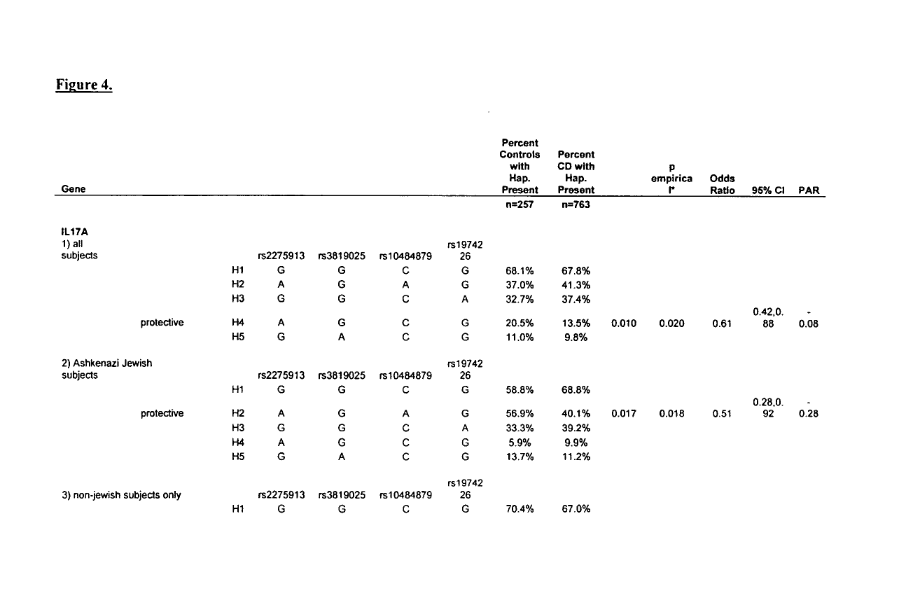

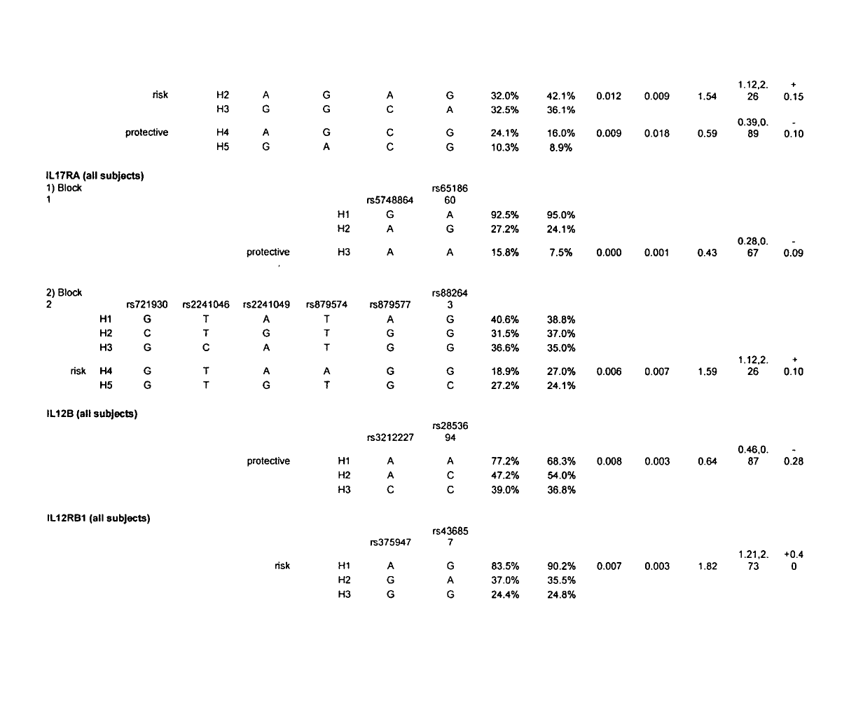

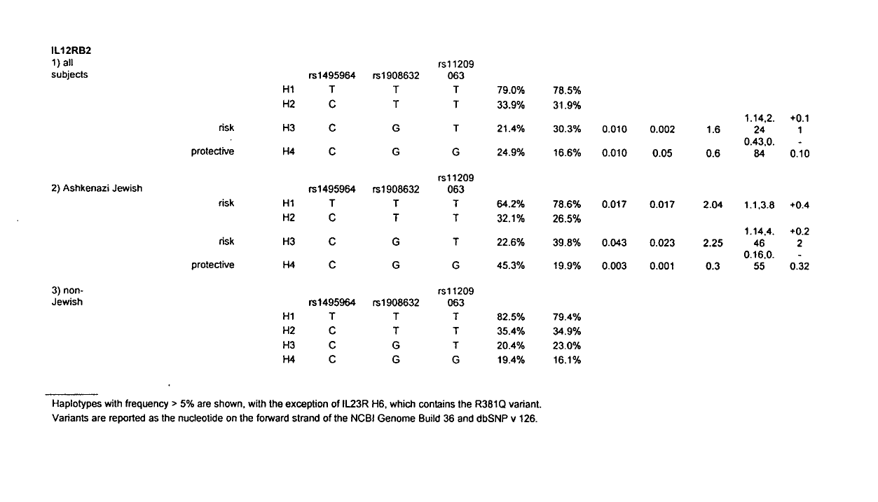

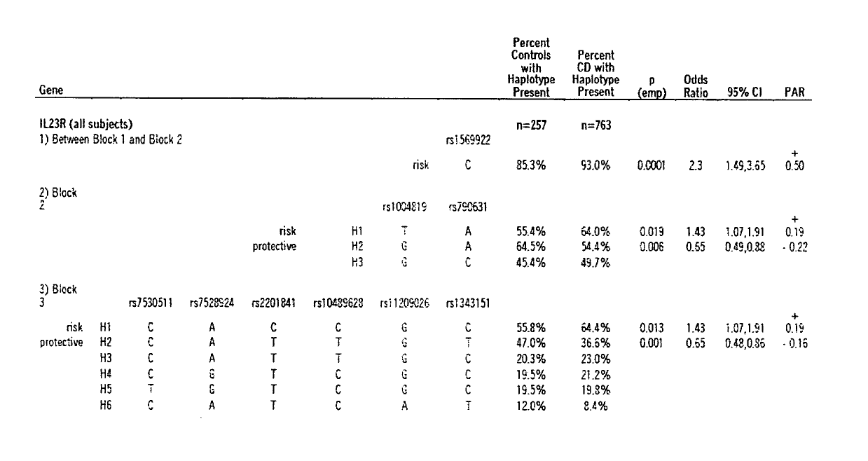

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“SNP” as used herein means single nucleotide polymorphism.

“Haplotype” as used herein refers to a set of single nucleotide polymorphisms (SNPs) on a gene or chromatid that are statistically associated.

“Risk variant” as used herein refers to an allele whose presence is associated with an increase in susceptibility to an inflammatory bowel disease, including but not limited to Crohn's Disease and ulcerative colitis, relative to an individual who does not have the risk variant.

“Protective variant” as used herein refers to an allele whose presence is associated with a low probability relative to a healthy individual of developing inflammatory bowel disease. The protective variant is more frequently present in healthy individuals compared to individuals diagnosed with inflammatory bowel disease.

“Risk haplotype” as used herein refers to a haplotype whose presence is associated with an increase in susceptibility to an inflammatory bowel disease, relative to an individual who does not have the risk haplotype.

“Protective haplotype” as used herein refers to a haplotype whose presence is associated with a low probability relative to a healthy individual of developing inflammatory bowel disease. The protective haplotype is more frequently present in healthy individuals compared to individuals diagnosed with inflammatory bowel disease.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, the abbreviation “IL12A” means interleukin 12A, “IL12B” means interleukin 12B, “IL12RB1” means interleukin 12 receptor beta 1, “IL12RB2” means interleukin 12 receptor beta 2, “IL17A” means interleukin 17A, “IL17RA” means interleukin 17 receptor A, “IL23A” means interleukin 23 alpha subunit p19, “IL23R” means interleukin 23 receptor.

As used herein, IL23R SNPs rs1569922, rs1004819, rs790631, rs2863212, rs7530511, rs7528924, rs2201841, rs11804284, rs10489628, rs11209026 and rs1343151, are also described herein as SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10 and SEQ. ID. NO.: 11, respectively. Examples of the IL23R genetic sequence are provided herein as SEQ. ID. NO.: 27 and SEQ. ID. NO.: 28.

As used herein, IL17A SNPs rs2275913, rs3819025, rs10484879 and rs1974226, are also described herein as SEQ. ID. NO.: 12, SEQ. ID. NO.: 13, SEQ. ID. NO.: 14 and SEQ. ID. NO.: 15, respectively. Examples of the IL17A genetic sequence are provided herein as SEQ. ID. NO.: 29 and SEQ. ID. NO.: 30.

As used herein, IL17RA SNPs rs721930, rs2241046, rs2241049, rs879574, rs879577 and rs882643, are also described herein as SEQ. ID. NO.: 16, SEQ. ID. NO.: 17, SEQ. ID. NO.: 18, SEQ. ID. NO.: 19, SEQ. ID. NO.: 20 and SEQ. ID. NO.: 21, respectively. Examples of the IL17RA genetic sequence are provided herein as SEQ. ID. NO.: 31 and SEQ. ID. NO.: 32.

As used herein, IL12RB1 SNPs rs375947 and rs436857, are also described herein as SEQ. ID. NO.: 22 and SEQ. ID. NO.: 23, respectively. Examples of the IL12RB1 genetic sequence are provided herein as SEQ. ID. NO.: 33, SEQ. ID. NO.: 34, SEQ. ID. NO.: 35 and SEQ. ID. NO.: 36.

As used herein, IL12RB2 SNPs rs1495964, rs1908632 and rs11209063, are also described herein as SEQ. ID. NO.: 24, SEQ. ID. NO.: 25 and SEQ. ID. NO.: 26, respectively. Examples of the IL12RB2 genetic sequence are provided herein as SEQ. ID. NO.: 32 and SEQ. ID. NO.: 33.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease. Other embodiments provide for methods of treating inflammatory bowel disease including but not limited to Crohn's Disease.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease, as described herein. The methods may also further include recording whether a genetic risk, susceptibility for inflammatory bowel disease including but not limited to Crohn's Disease exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors tested the hypothesis that risk haplotypes in genes of the IL23/IL17 pathway contribute to increased susceptibility for CD. 763 CD subjects and 254 controls were genotyped for single nucleotide polymorphisms in the IL23A, IL23R, IL17A, IL17RA, IL12A, IL12B, IL12RB1, and IL12RB2 genes. Genotyping was performed using both Illumina bead array and ABI TaqMan MGB technologies. Common haplotypes, with control frequencies greater than 5%, were assigned using Phase v2 and were tested for association with CD by chi square, with significance assessed using permutation.

As further disclosed herein, the inventors found that haplotypes with increased risk for CD were observed in the IL23R, IL17A, IL17RA genes, and IL12RB1 genes (IL23R, 55% control, 64% CD, p=0.015; IL17A, 32% control, 36% CD, p=0.015; IL17A, 19% control, 27% CD, p=0.003; IL12RB1, 84% control, 90% CD, p=0.004). These haplotypes substantially increase CD risk as seen by a large estimated population attributable risk (PAR, IL23R risk, ˜19%; IL17A risk, ˜16%; IL17RA risk, ˜10%). The odds ratio for CD increased with the number of risk haplotypes from these 4 genes (OR=1 for 0 or 1 risk haplotype, 1.3 for 2, 2.5 for 3, and 4 for 4 risk haplotypes, p<0.0001). Furthermore, a synergy was observed between IL23R and IL17A, and between IL23R and IL17RA, in that an increased odds ratio (OR) for CD was observed when a risk haplotype from both genes was present (OR ˜1 for the presence of the risk haplotype from IL23R or IL17A and 2.4 for both, p=0.047 for interaction; OR ˜1.1 for IL23R or IL17RA and ˜3 for both, p=0.036 for interaction). Similarly, no interaction between any of the genes tested and NOD2/CARD15 mutations was observed.

In one embodiment, the present invention provides methods of diagnosing and/or predicting susceptibility to Crohn's Disease by determining the presence or absence of risk haplotypes in IL23R, IL17A, and/or IL17RA genes. In another embodiment, the present invention provides methods of prognosis of Crohn's Disease by determining the presence or absence of risk haplotypes in IL23R, IL17A, and/or IL17RA genes. In another embodiment, the present invention provides methods of treatment of Crohn's Disease by inhibiting the IL23/IL17 pathway.

As disclosed herein, for IL12B, the tagSNPs formed one haplotype block and H1 was associated with a modestly decreased susceptibility for CD (“protective,”Controls, 77.2%, CD 68.3%, p=0.004) and a population attributable risk of minus ˜28%. For IL12RB1, the tagSNPs formed one haplotype block and H1 was associated with a greater susceptibility for CD (“risk,” Control, 83.5%, CD, 90.2%, p=0.004). For IL12RB2, the tagSNPs formed one haplotype block and H4 was associated with a decrease in susceptibility for CD (“protective,” Control, 24.3%, CD, 18.5%, p=0.036). In contrast to the other observed associations, this association of CD and IL12RB2 haplotypes was particular to Ashkenazi Jewish subjects because when Ashkenazi Jewish and non-Jewish CD subjects were analyzed separately, the association of CD and the IL12RB2H4 protective haplotype was observed in the Jewish subjects only (Jewish: Control. 43.4%, CD 21.9%, p=0.001; non-Jewish: Control, 19.4%, CD, 16.1%, p is not significant). Furthermore, a significant risk haplotype for this population was also observed, the presence of IL12RB2H1 (Jewish: Control, 62.3%, CD, 78.6%, p=0.009; non-Jewish: Control, 82.5%, CD, 79.4%, p not significant).

In one embodiment, the present invention provides methods of diagnosing and/or predicting susceptibility to Crohn's Disease in an individual by determining the presence or absence of H1 susceptibility haplotype of IL12RB1 in the individual. In another embodiment, the present invention provides method of treatment of Crohn's Disease in an individual by inhibiting the expression of H1 susceptibility haplotype of IL12RB1 in the individual. In another embodiment, the present invention provides method of treatment of Crohn's Disease by determining the presence or absence of H1 susceptibility haplotype of IL12RB1 and treating the Crohn's Disease.

In one embodiment, the present invention provides methods of diagnosing and/or predicting susceptibility to Crohn's Disease in a Jewish individual by determining the presence or absence of H1 susceptibility haplotype of IL12RB2 in the Jewish individual. In another embodiment, the present invention provides methods of treatment of Crohn's Disease by inhibiting the expression of H1 susceptibility haplotype of IL12RB2 in the Jewish individual. In another embodiment, the present invention provides method of treatment of Crohn's Disease by determining the presence of H1 susceptibility haplotype of IL12RB2 in the Jewish individual and treating the Crohn's Disease.

In one embodiment, the present invention provides methods of diagnosing and/or predicting susceptibility to Crohn's Disease in an individual by determining the presence or absence of H3 susceptibility haplotype of IL12RB2 in the individual. In another embodiment, the present invention provides methods of treatment of Crohn's Disease by inhibiting the expression of H3 susceptibility haplotype of IL12RB2 in the individual. In another embodiment, the present invention provides method of treatment of Crohn's Disease by determining the presence of H3 susceptibility haplotype of IL12RB2 in the individual and treating the Crohn's Disease.

In one embodiment, the present invention provides methods of diagnosing and/or predicting protection against Crohn's Disease in a Jewish individual by determining the presence or absence of H4 protective haplotype of IL12RB2 in the individual. In another embodiment, the present invention provides methods of prognosis of Crohn's Disease in an individual by determining the presence or absence of H4 protective haplotype of IL12RB2 in the individual. In another embodiment, the present invention provides methods of treatment of Crohn's Disease in an individual by inhibiting the expression of H4 protective haplotype of IL12RB2 in the individual.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a genetic variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI).

Sequence analysis may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### IL23/IL17 Pathway Genes and Their Interactions Provide Major Genetic Susceptibility to Crohn's Disease

The inventors tested the hypothesis that haplotypes in genes of the IL23/IL17 pathway contribute to increased susceptibility for CD. 763 CD subjects and 254 controls were genotyped for single nucleotide polymorphisms in the IL23A, IL23R, IL17A, IL17RA, IL12A, IL12B, IL12RB1, and IL12RB2 genes. Genotyping was performed using both Illumina bead array and ABI TaqMan MGB technologies. Common haplotypes, with control frequencies greater than 5%, were assigned using Phase v2 and were tested for association with CD by chi square, with significance assessed using permutation.

The inventors found that haplotypes with increased risk for CD were observed in the IL23R, IL17A, IL17RA genes, and IL12RB1 genes (IL23R, 55% control, 64% CD, p=0.015; IL17A, 32% control, 36% CD, p=0.015; IL17A, 19% control, 27% CD, p=0.003; IL12RB1, 84% control, 90% CD, p=0.004). These haplotypes substantially increase CD risk as seen by a large estimated population attributable risk (PAR, IL23R risk, ˜19%; IL17A risk, ˜16%; IL17RA risk, ˜10%; IL12RB1 risk). The odds ratio for CD increased with the number of risk haplotypes from these 4 genes (OR=1 for 0 or 1 risk haplotype, 1.3 for 2, 2.5 for 3, and 4 for 4 risk haplotypes, p<0.0001). Furthermore, a synergy was observed between IL23R and IL17A, and between IL23R and IL17RA, in that an increased odds ratio (OR) for CD was observed when a risk haplotype from both genes was present (OR ˜1 for the presence of the risk haplotype from IL23R or IL17A and 2.4 for both, p=0.047 for interaction; OR ˜1.1 for IL23R or IL17RA and ˜3 for both, p=0.036 for interaction). Similarly, no interaction between any of the genes tested and NOD2/CARD15 mutations was observed.

The identification of an IL23R risk haplotype with high population frequency and large population attributable risk demonstrates the importance of this gene for CD susceptibility. The observations of associations between CD and IL17A, IL17RA, and IL12RB1 haplotypes suggests that the IL23/IL17 pathway is important for CD pathogenesis and may be a target for therapy. The lack of interaction of IL23/IL17-related risk variants with NOD2/CARD15 mutations suggest that the IL23/IL17 pathway and NOD2/CARD15 act separately to promote CD.

### Example 2

### Subjects

Recruitment of subjects at the Cedars-Sinai Medical Center Inflammatory Bowel Disease center was conducted under the approval of the Cedars-Sinai Medical Center Institutional Review Board. Disease phenotype was assigned using a combination of standard endoscopic, histological, and radiographic features. Ashkenazi Jewish ethnicity was assigned when two or more grandparents were of Ashkenazi Jewish origin.

### Example 3

### Selection of SNPs

SNPs were selected by applying the “Tagger” option in the program Haploview to data from the International HapMap Project. SNPs that “tagged” major Caucasian haplotypes and at the same time that were predicted to be compatible with the Illumina genotyping technology using the Illumina Assay Design Tool were genotyped in the initial phases of this study. Since the inventors were interested in major genetic effects for this study rather than rare alleles, the goal of “tagging” was to find a set of tagSNPs in linkage disequilibrium with all SNPs in the HapMap data with a minor allele frequency ≧5%; in some cases this goal was not completely met due to the limitations of the Illumina technology. A few SNPs were also added that were: 1) non-synonymous and had a minor allele frequency greater than 3%, 2) redundant in order to accommodate some assay failure in the initial Illumina run, and 3) markers suggested by information provided by SeattleSNPs. SNPs showing positive associations were selected for further genotyping by ABI technology.

### Example 4

TABLE 1
SNPs Genotyped

| | | | Percent with Minor Allele | | |
|---|---|---|---|---|---|
| | | | Controls | CD | |
| dbSNP | Gene | TaqMan Assay if used | N = 257 | N = 753 | p-value |
| rs2853694 | IL12B, p40 | C_2084298_10 | 77.3 | 67.9 | 0.0042 |
| rs3212227 | IL12B, p40 | C_2084293_10 | 40.5 | 38.2 | |
| rs3213096 | IL12B, p40 | | 0.8 | 1.6 | |
| rs3213119 | IL12B, p40 | | 2.8 | 4.6 | |
| rs375947 | IL12RB1 | | 55.1 | 56.2 | |
| rs376008 | IL12RB1 | C_795459_10 | 54.7 | 56.4 | |
| rs425648 | IL12RB1 | | 37.6 | 35.8 | |
| rs436857 | IL12RB1 | | 37.6 | 35.7 | |
| rs438421 | IL12RB1 | C_795437_10 | 40.5 | 50.0 | 0.01 |
| rs10484879 | IL17A | custom design | 38.9 | 43.5 | |
| rs1892280 | IL17A | C_12029406_10 | 42.7 | 52.0 | 0.01 |
| rs1974226 | IL17A | custom design | 32.0 | 39.2 | 0.04 |
| rs2275913 | IL17A | C_15879983_10 | 52.8 | 52.0 | |
| rs2894798 | IL17A | | 44.9 | 52.6 | 0.034 |
| rs3819024 | IL17A | | 56.9 | 56.0 | |
| rs3819025 | IL17A | C_292276_10 | 11.2 | 10.3 | |
| rs4711998 | IL17A | | 45.7 | 40.2 | |
| rs7747909 | IL17A | custom design | 39.4 | 44.4 | |
| rs8193036 | IL17A | | 40.9 | 40.9 | |
| rs2041629 | IL17RA | | 30.7 | 34.4 | |
| rs2241042 | IL17RA | | 60.6 | 65.1 | |
| rs2241046 | IL17RA | C_2666438_1_{—} | 36.9 | 36.0 | |
| rs2241048 | IL17RA | | 57.0 | 78.0 | <0.0001 |
| rs2241049 | IL17RA | custom design | 57.1 | 60.1 | |
| rs2302519 | IL17RA | C_15757768_10 | 88.5 | 65.1 | |
| rs5518660 | IL17RA | | 27.6 | 32.2 | |
| rs721930 | IL17RA | C_12689_10 | 33.1 | 38.2 | |
| rs7288159 | IL17RA | | 41.7 | 37.2 | |
| rs879574 | IL17RA | C_11283754_10 | 21.7 | 30.8 | 0.005 |
| rs879575 | IL17RA | C_7620883_10 | 44.4 | 41.2 | |
| rs879577 | IL17RA | C_2666446_20 | 44.9 | 42.4 | |
| rs882643 | IL17RA | C_7620881_10 | 28.4 | 24.6 | |
| rs887796 | IL17RA | | 33.9 | 32.5 | |
| rs9606603 | IL17RA | | 70.9 | 72.4 | |
| rs11171806 | IL23A, p19 | C_25985467_10 | 10.7 | 10.8 | |
| rs1004819 | IL23R | C_1272321_10 | 55.4 | 64.0 | 0.015 |
| rs10489628 | IL23R | C_11283754_10 | 83.4 | 56.1 | 0.045 |
| rs11209008 | IL23R | | 7.6 | 4.2 | 0.032 |
| rs11465797 | IL23R | | 11.9 | 8.6 | |
| rs11804284 | IL23R | C_2990003_10 | 19.5 | 20.2 | |
| rs12041056 | IL23R | | 19.6 | 17.7 | |
| rs1343151 | IL23R | C_8367043_10 | 57.7 | 45.2 | |
| rs1589922 | IL23R | | 14.7 | 7.0 | 0.0000 |
| rs1884444 | IL23R | | 69.7 | 68.0 | |
| rs2201841 | IL23R | | 56.8 | 64.4 | 0.014 |
| rs2863212 | IL23R | | 16.7 | 16.7 | |
| rs6671221 | IL23R | | 89.7 | 88.0 | |
| rs7528924 | IL23R | C_2990015_10 | 37.5 | 39.5 | |
| rs7530511 | IL23R | C_2990018_10 | 19.9 | 20.8 | |
| rs790631 | IL23R | C_1272311_10 | 45.4 | 49.6 | |

### Example 5

### Genotyping

DNA was isolated from Epstein Barr virus transformed lymphoblastoid cell lines using proteinase K digestion, organic extraction, and ethanol precipitation. Single nucleotide markers (SNPs) were genotyped using one of two methods: (1) the oligonucleotide ligation assay, Illumina Golden Gate technology, following the manufacturer's protocol (Illumina, San Diego, Calif.), and (2) the 5′-extension reaction, TaqMan MGB technology, following the manufacturer's protocol (Applied Biosystems, Bulletin #4322856). Consistency of SNP genotyping between the two methods was checked for each SNP by genotyping 100 samples with both methods.

### Example 5

### Statistical Analyses

Haplotype blocks were determined using the “Tagger” routine of the program Haploview. Haplotypes of subjects were inferred from the genotyping data using the program PHASE v2. The association of the presence of a haplotype was tested using the chi-square test and the significance of results was assessed by applying a permutation test to the data in order to correct for multiple testing due to the number of haplotypes. Results with significance were defined by p<0.05 by permutation test. Due to sample size considerations, the results reported are for all CD and control subjects with Jewish and non-Jewish subjects combined. The notable exception to this is that an IL17A “risk” haplotype specific to the non-Jewish population was identified in the hypothesis-generating phase of this study and used for subsequent gene-gene interaction studies. Population attributable risk was estimated by assuming that 1) the frequency of a particular haplotype in the controls reflected the population frequency of that haplotype, and 2) the odds ratio for the association of a given haplotype reflected the relative risk of that haplotype for Crohn's disease. For this report, haplotypes are numbered in order of frequency in controls (H1, H2, and so forth) and the nucleotides for each tagSNP are listed in Table 1 according to the forward strand of the NCBI human genome build 36 and dbSNP. A “major” haplotype in this report is a haplotype with a population frequency greater than 5% in the controls.

### Example 6

### TagSNPs Selected in Genes Related to the IL12/IL23 Pathway

TagSNPs were first selected for the major Caucasian haplotypes in eight genes related to the IL12/IL23 pathway (Table 2), genotyped in a CD case-control cohort, used to infer haplotypes, and then tested for association with Crohn's disease.

TABLE 2

| GENE | GENE | |
|---|---|---|
| ID* | ABBREVIATION | GENE DESCRIPTION |
| 3592 | IL12A | Interleukin 12A (natural killer cell |
| | | stimulatory factor 1, cytotoxic |
| | | lymphocyte maturation factor 1, p35) |
| 3593 | IL12B | Interleukin 12B (natural killer cell |
| | | stimulatory factor 2, cytotoxic |
| | | lymphocyte maturation factor 2, p40) |
| 3594 | IL12RB1 | Interleukin 12 receptor beta 1 |
| 3595 | IL12RB2 | Interleukin 12 receptor beta 2 |
| 3605 | IL17A | Interleukin 17A |
| 23765 | IL17RA | Interleukin 17 receptor A |
| 51561 | IL23A | Interleukin 23 alpha subunit p19 |
| 148233 | IL23R | Interleukin 23 receptor |

*Gene ID from dbGene of the National Center for Biotechnology Information, National Library of Medicine, National Institutes of Health.

### Example 7

### IL23R

IL23R haplotypes with high population frequency were observed to be associated with CD. Three IL23R haplotype blocks were inferred from tagSNP data. No associations between CD and IL23R Block 1 haplotypes were observed. However, CD was associated with the individual SNP rs1569922, located between Block 1 and Block 2 (85% in controls compared with 93% in CD subjects, p<0.0001) as well as haplotypes in blocks 2 and 3. Haplotypes that both increased CD risk (“risk,” IL23R Block 2H1 and IL23R Block 3H1) and decreased CD risk (“protective,” IL23R Block 2H2 and IL23R Block 3H2) were observed. Furthermore, within each block, the odds ratio for CD was increased with the number of copies of the “risk” haplotype and was decreased with the number of copies of the “protective” haplotype from 0 through 1 to copies (“risk:” IL23R Block 2H1, p(trend)=0.0091, IL23R Block 3H1, p(trend)=0.0097; “protective:” IL23R Block 2H2, p(trend)=0.0002, Block 2H2, p(trend)=0.0011). The odds ratio for CD risk was increased with the number of “risk” haplotypes from both haplotype blocks (“risk,” p(trend)=0.0072; “protective,” p(trend)<0.0001). In this study, the IL23R functional and “protective” allele (R381Q, rs11209026) was located on IL23R Block 3H6. The magnitude of the population attributable risk, or the amount of the disease that would not exist if a risk factor is removed from a population, was much greater for the presence of the “risk” or the “protective” haplotypes reported here than for the previously reported IL23R Block 3H6 containing IL23R R381Q (˜20% for the presence of either the “risk” haplotypes together or the “protective” haplotypes together and ˜4% for the presence of Block 3H6).

### Example 8

### IL17A

The tagSNPs formed one haplotype block spanning most of this gene. When all subjects were considered, IL17A H4 was “protective,” conferring a decreased risk for CD (frequency in controls 20.5%, in CD 13.5%, p=0.007). When only non-Jewish subjects were considered, IL17A H4 remained “protective” (Controls, 24.1%, CD 16.0%, p=0.014) and IL17A H2 was a “risk” haplotype, conferring increased risk for CD (Controls, 32.0%, CD, 42.1%, p=0.015). These IL17A haplotypes were associated with a substantial risk for CD in non-Jewish subjects; the magnitude of the population attributable risk was ˜16% for IL17A H2 (“risk”) in non-Jewish subjects and minus ˜10% for IL17A H4 (“protective”).

### Example 9

### IL17RA

The tagSNPs formed two haplotype blocks. IL17RA Block 1H3 was associated with a decreased susceptibility for CD (“protective,” Controls 15.8%, CD 7.5%, p<0.0000) and IL17RA Block 2H4 was associated with an increased susceptibility for CD (Controls, 18.9%, CD, 27.0%, p=0.01). The magnitude of the population attributable risk for IL17RA Block 2H4 was ˜10% and for Block 1H3 was minus ˜3%.

### Example 10

### IL12B, IL12RB1, IL12RB2

For IL12B, the tagSNPs formed one haplotype block and H1 was associated with a modestly decreased susceptibility for CD (“protective,” Controls, 77.2%, CD 68.3%, p=0.004) and a population attributable risk of minus ˜28%. For IL12RB1, the tagSNPs formed one haplotype block and H1 was associated with a greater susceptibility for CD (“risk,” Control, 83.5%, CD, 90.2%, p=0.004). For IL12RB2, the tagSNPs formed one haplotype block and H4 was associated with a modest decrease in susceptibility for CD (“protective,” Control, 24.3%, CD, 18.5%, p=0.036). In contrast to the other observed associations, this association of CD and IL12RB2 haplotypes was particular to Ashkenazi Jewish subjects because when Ashkenazi Jewish and non-Jewish CD subjects were analyzed separately, the association of CD and the IL12RB2H4 protective haplotype was observed in the Jewish subjects only (Jewish: Control. 43.4%, CD 21.9%, p=0.001; non-Jewish: Control, 19.4%, CD, 16.1%, p is not significant). Furthermore, a significant risk haplotype for this population was also observed, the presence of IL12RB2H1 (Jewish: Control, 62.3%, CD, 78.6%, p=0.009; non-Jewish: Control, 82.5%, CD, 79.4%, p not significant).

### Example 11

### IL12A (p35) and IL23 (p19)

No association was observed between CD and haplotypes formed by 4 IL12A tagSNP's nor between IL23 tagSNP rs11171806.

### Example 12

### Interactions Between IL23R, 117A, IL17RA, and IL12RB1—Table 3

One hypothesis was that the combination of variation in genes related to the IL23/IL17 pathway contribute to increased risk of CD. Therefore the inventors analyzed combinations of the risk and protective haplotypes observed to increase CD susceptibility individually (Table 3).

TABLE 3 (a)-(b)
Gene-Gene Interactions Between IL23R, IL17A, and IL17RA
(a)

| Presence | | | | | | | |
|---|---|---|---|---|---|---|---|
| of IL23R | | | | | | | |
| Block 2 | | | | | | | |
| H1 or | | | | | | | |
| IL23R | Presence | | | | | Mantel- | |
| Block 3 | of IL17A | | | Odds | | Haenszel P | Interaction P |
| H1 | H2 | CD | Control | Ratio | 95% CI | value | value |
| No | No | 90 | 52 | 1 | | 0.0017 | 0.047 |
| No | Yes | 52 | 30 | 1.0 | 0.6-1.8 | | |
| Yes | No | 166 | 84 | 1.1 | 0.7-1.8 | | |
| Yes | Yes | 133 | 32 | 2.4 | 1.4-4.0 | | |

(b)

| Presence | | | | | | | |
|---|---|---|---|---|---|---|---|
| of IL23R | | | | | | | |
| Block 2 | | | | | | | |
| H1 or | | | | | | | |
| IL23R | Presence | | | | | Mantel- | |
| Block 3 | of IL17RA | | | Odds | | Haenszel | Inter-P |
| H1 | H4 | CD | Control | Ratio | 95% CI | P value | value |
| No | No | 175 | 78 | 1 | | 0.0003 | 0.036 |
| No | Yes | 65 | 27 | 1.1 | 0.6-1.8 | | |
| Yes | No | 370 | 126 | 1.3 | 0.9-1.8 | | |
| Yes | Yes | 138 | 20 | 3.0 | 1.8-5.2 | | |

First, IL23R and IL17A variation interacted to increase CD susceptibility. When the IL23R and IL17A “risk” haplotypes were present together, the odds ratio for CD increased substantially over the odds ratio for CD when either “risk” haplotype was present alone (IL23R H1 from either Block 2 or 3 present and IL17A H4 present, OR=2.4, compared with 1.0-1.1 when either one “risk” haplotype was present or no “risk” haplotype was present, p(Mantel-Haenszel)=0.0017, p(logistic regression test for interaction)=0.047). The Mantel-Haenszel analysis suggested that the trend from no “risk” haplotypes through one to two is significant while the logistic regression analysis for interaction suggested that the two risk haplotypes synergistically interacted to increase CD susceptibility.

Second, IL23R and IL17RA variation also interacted to increase CD susceptibility. When the IL23R and IL17RA risk haplotypes were present together, the odds ratio for CD increased over the odds ratio when either “risk” haplotype was present alone (IL23R H1 from either Block 2 or 3 present and IL17RA H4 present, OR=3.0, compared with 1.0-1.3 when either one “risk” haplotype or no “risk” haplotype was present, p(Mantel-Haenszel)=0.0003, p(logistic regression for interaction)=0.036). Again, the Mantel-Haenszel analysis suggested that the trend from no “risk” haplotype through one to two is significant while the logistic regression analysis suggested that the two risk haplotypes synergistically interacted to increase CD susceptibility.

Third, but in contrast, IL17A and IL17RA variation was additive for each but with no interaction. The odds ratio for CD when both IL17A and IL17RA “risk” haplotypes were present was not greater than the odds ratio for CD when only one of the IL17A or IL17RA “risk” haplotypes was present (IL17A H2 and IL17RA H4 present, OR=1.7, either IL17A H2 or IL17RA H4 present, OR=1.5-1.7, no IL17A or IL17RA “risk” haplotype present, OR=1.0, p(Mantel-Haenszel)=0.005, p(logistic regression for interaction) was not significant). The Mantel-Haenszel analysis suggested that the presence of either IL17A or IL17RA “risk” haplotype significantly increased CD susceptibility, but the non-significant logistic regression analysis suggested that variants in these two genes were not interacting to increase CD susceptibility.

Combining the risk haplotypes from IL23R, IL17A, IL17RA, and IL12RB1 in a single analysis showed a significant increase in the odds ratio for CD from no “risk” haplotype to 3 “risk” haplotypes (OR for CD is 1, 1.1, 1.3, 2.5, and 3.7 for 0, 1, 2, 3, 4 “risk” haplotypes, respectively, p(Mantel-Haenszel)<0.0001). This analysis demonstrated that IL23R, IL17A, IL17RA, and IL12RB1 genetic variation contributes substantially to CD susceptibility.

### Example 13

### Interaction With CARD15 Mutations

Since a recent genome-wide association study observed that CARD15 and IL23R were the two greatest contributors to CD risk, the interaction between three common CARD15 mutations and IL23/IL17 haplotypes was examined (Table 4). CD susceptibility was significantly increased when one CARD15 “risk” mutation was present with one of the IL23R, IL17A, and IL17RA “risk” haplotypes (p-values for Mantel-Haenszel tests were significant). However, when tested for interaction, the presence of a CARD15 mutation did not interact with the presence of one of the IL23R, IL17A or IL17RA “risk” haplotypes (p-values for the interaction test were not significant).

TABLE 4
Interactions between CARD15 mutations and IL23R, IL17A, and IL17RA
“risk” haplotypes
a) CARD15 and IL23R in all subjects

| Presence of at | Presence of | | | | | | |
|---|---|---|---|---|---|---|---|
| least 1 | IL23R Block 2 H1 | | | | 95% | Mantel- | |
| CARD15 | or IL23R Block 3 | | | Odds | Confidence | Haenszel | Interaction P |
| mutation* | H1 | CD | Control | Ratio | Interval | P value | value |
| No | No | 150 | 97 | 1 | | <0.0001 | 0.07 |
| Yes | No | 90 | 8 | 7.3 | 3.4-15.7 | | |
| No | Yes | 339 | 126 | 1.7 | 1.3-2.4 | | |
| Yes | Yes | 167 | 20 | 5.4 | 3.2-9.2 | | |

b) CARD15 and IL17A in non-Jewish subjects

| Presence of at | | | | | | | |
|---|---|---|---|---|---|---|---|
| least 1 | | | | | 95% | Mantel- | |
| CARD15 | Presence of | | | Odds | Confidence | Haenszel | Interaction P |
| mutation | IL17A H2 | CD | Control | Ratio | Interval | P value | value |
| No | No | 159 | 120 | 1 | | <0.0001 | 0.5 |
| Yes | No | 101 | 18 | 4.2 | 2.4-7.4 | | |
| No | Yes | 131 | 59 | 1.7 | 1.1-2.5 | | |
| Yes | Yes | 58 | 4 | 10.9 | 3.9-31 | | |

c) CARD15 and IL17R in all subjects

| Presence of at | | | | | | | |
|---|---|---|---|---|---|---|---|
| least 1 | | | | | 95% | Mantel- | |
| CARD15 | Presence of | | | Odds | Confidence | Haenszel | Interaction P |
| mutation | IL17A H2 | CD | Control | Ratio | Interval | P value | value |
| No | No | 361 | 181 | 1 | | <0.0001 | 0.6 |
| Yes | No | 196 | 25 | 3.9 | 2.5-6.2 | | |
| No | Yes | 140 | 44 | 1.6 | 1.1-3.2 | | |
| Yes | Yes | 66 | 4 | 8.3 | 3.0-23.0 | | |

*CARD15 mutations are commonly known as SNP8 (CARD15 R702W; rs2066844), SNP12 (G908R; rs2068845), and SNP13 (L1007fsinsC; rs2066847).

### Example 14

### Role of Th17 cell in Crohn's Disease Pathogenesis—Table 5

The significant genetic associations and high population attributable risks reported here support the hypothesis that genes in the IL23R/IL17 pathway, individually and in interaction, are major contributors to the genetic susceptibility of Crohn's disease (CD). Since increasing evidence implicates this pathway in the proliferation and subsequent action of the Th17 cell, these results suggest a role for this cell type in CD pathogenesis.

The association of CD with ten IL23R single nucleotide polymorphisms (SNPs), in particular rs11209026 (Arg381GIn), was observed in a whole genome association study of ileal CD; the inventors have confirmed this finding in a pediatric cohort. These observations support the concept that the IL23R gene is a genetic determinant of CD. However, based on the low frequency of the minor allele of IL23R Arg381Gln in the general population, the population attributable risk (PAR) for this allele would be on the order of ˜4% (Control, 7%, CD 1.9%). The “risk” and “protective” IL23R haplotypes reported here are at a much higher frequency in the general population, substantially raising the estimate of the PAR for the IL23R gene to the order of ˜20%. These considerations support the concept that the IL23R gene is a major genetic determinant of CD, on the order of the presence of a CARD15/NOD2 mutation (Table 5)

TABLE 5
Cumulative Population Attributable Risk (PAR) for CD
a) Odds Ratio and Population Attributable Risk
for Carriers of Risk Haplotype

| Gene | OR | PAR |
|---|---|---|
| IL23R | 1.5 | 23 |
| IL17A | 1.6 | 16 |
| IL17RA | 1.6 | 16 |
| NOD2 | 4.2 | 26 |

b) Population Attributable Risk for Carriers of
Two Risk Haplotypes, Pairwise

PAR

| | | Risk | |
|---|---|---|---|
| | | Haplotype | Risk |
| | | from Either | Haplotype |
| | | or Both | from Both |
| | | Genes | Genes |
| Gene 1 | Gene 2 | Present | Present |
| IL23R | IL17A | 22 | 15 |
| IL23R | IL17RA | 25 | 8 |
| IL23R | NOD2 | 48 | 1 |
| IL17A | IL17RA | 21 | 0.1 |
| IL17A | NOD2 | 41 | 2 |
| IL17RA | NOD2 | 34 | 2 |

In addition to IL23R, associations were also observed between CD and common haplotypes in other genes in the IL23/IL17 pathway: IL17A, IL17RA, IL12B, and IL12RB1. A “risk” haplotype, conferring a greater susceptibility to CD, or a “protective” haplotype, conferring a reduced susceptibility to CD, or both, was observed in each of these genes. Both were observed with IL17A and with IL17RA in non-Jewish CD subjects, with PAR on the order of ˜10%; an IL12B protective haplotype was also observed with PAR on the order of ˜28%. Furthermore, risk haplotypes of IL23R and IL17A and of IL23R and IL17RA interacted to increase CD risk only when both were present, supporting the concept that CD pathophysiology involves the products of these genes together. Further support for this concept was the observation of increasing odds ratio for CD as the risk haplotypes for these genes were combined.

While additive to increase CD risk, no interaction between mutations in CARD15/NOD2 and risk haplotypes in the IL23/IL17 pathway were observed. This observation suggests that CARD15/NOD2 and IL23/IL17 variants define two separate pathways to intestinal inflammation. Extensive work with mouse models of intestinal inflammation, developed by “knocking out” many different immune-related genes have demonstrated that there are multiple genetic pathways to intestinal inflammation. If so, then variation in IL23/IL17 related genes may be useful to distinguish CD subtypes with different underlying pathophysiological mechanisms, and suggests that therapies targeted at IL23/IL17 successfully treat IL23/IL17 pathway-related CD subtypes.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive. One skilled in the art will recognize many methods and materials similar or equivalent to those described herein, which could be used in the practice of the present invention. Indeed, the present invention is in no way limited to the methods and materials described. Furthermore, one of skill in the art would recognize that the invention can be applied to various inflammatory conditions and disorders and autoimmune diseases besides that of inflammatory bowel disease. It will also be readily apparent to one of skill in the art that the invention can be used in conjunction with a variety of phenotypes, such as serological markers, additional genetic variants, biochemical markers, abnormally expressed biological pathways, and variable clinical manifestations.

1. A method of diagnosing susceptibility to Crohn's Disease in an individual, comprising:
determining the presence or absence of a first risk haplotype at the IL23R locus, the presence or absence of a second risk haplotype at the IL17A locus, the presence or absence of a third risk haplotype at the IL17RA locus, and the presence or absence of a fourth risk haplotype at the IL12RB1 locus,
wherein the presence of four of said risk haplotypes presents a greater susceptibility than the presence of three, two, one or none of said risk haplotypes, and the presence of three risk haplotypes presents a greater susceptibility than the presence of two, one or none of said risk haplotypes, and the presence of two risk haplotypes presents a greater susceptibility than the presence of one or none of said risk haplotypes, and the presence of one of said risk haplotypes presents a greater susceptibility than the presence of none of said risk haplotypes.

2. The method of claim 1, wherein the first risk haplotype at the IL23R locus comprises IL23R Block 2H1 and/or Block 3H1.

3. The method of claim 1, wherein the first risk haplotype at the IL23R locus comprises a variant selected from the group consisting of SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID NO.: 6, SEQ. ID NO.: 7, SEQ. ID. NO.: 8, SEQ. ID. NO.: 9, SEQ. ID. NO.: 10 and SEQ. ID. NO.: 11.

4. The method of claim 1, wherein the second risk haplotype at the IL17A locus comprises IL17A H2.

5. The method of claim 1, wherein the second risk haplotype at the IL17A locus comprises a variant selected from the group consisting of SEQ. ID. NO.: 12, SEQ. ID. NO.: 13, SEQ. ID. NO.: 14 and SEQ. ID. NO.: 15.

6. The method of claim 1, wherein the third risk haplotype at the IL17RA locus comprises IL17RA Block 2H4.

7. The method of claim 1, wherein the third risk haplotype at the IL17RA locus comprises a variant selected from the group consisting of SEQ. ID. NO.: 16, SEQ. ID. NO.: 17, SEQ. ID. NO.: 18, SEQ. ID. NO.: 19, SEQ. ID. NO.: 20 and SEQ. ID. NO.: 21.

8. The method of claim 1, wherein the fourth risk haplotype at the IL12RB1 locus comprises IL12RB1H1.

9. The method of claim 1, wherein the fourth risk haplotype at the IL12RB1 locus comprises a variant selected from the group consisting of SEQ. ID. NO.: 22 and SEQ. ID. NO.: 23.

10. A method of treating Crohn's Disease, comprising:
determining the presence of one or more risk haplotypes at the IL12RB1 locus; and
treating the Crohn's Disease.

11. The method of claim 10, wherein one of said one or more risk haplotypes at the IL12RB1 locus comprises SEQ. ID. NO.: 22 and SEQ. ID. NO.: 23.

12. A method of determining a low probability relative to a healthy subject of developing Crohn's Disease, comprising:
determining the presence or absence of a protective haplotype at the IL12RB2 locus in the individual; and
diagnosing a low probability of developing Crohn's Disease, relative to a healthy subject, based upon the presence of the protective haplotype at the IL12RB2 locus.

13. The method of claim 12, wherein the protective haplotype at the IL12RB2 locus comprises IL12RB2H4.

14. The method of claim 12, wherein the protective haplotype at the IL12RB2 locus comprises SEQ. ID. NO.: 24, SEQ. ID. NO.: 25 and SEQ. ID. NO.: 26.

15. The method of claim 12, wherein the individual is Ashkenazi Jewish.

16. A method of diagnosing susceptibility to Crohn's Disease in an individual, comprising:
determining the presence or absence of one or more risk haplotypes at the IL12RB2 locus in the individual; and
diagnosing susceptibility to Crohn's Disease based upon the presence of one or more risk haplotypes at the IL12RB2 locus.

17. The method of claim 16, wherein one of said one or more risk haplotypes at the IL12RB2 locus is H3.

18. The method of claim 16, wherein one of said one or more risk haplotypes at the IL12RB2 locus is H1.

19. The method of claim 18, wherein the individual is Ashkenazi Jewish.

20. The method of claim 16, wherein one of said one or more risk haplotypes at the IL12RB2 locus comprises SEQ. ID. NO.: 24, SEQ. ID. NO.: 25 and SEQ. ID. NO.: 26.

21. A method of treating Crohn's Disease, comprising:
determining the presence of one or more risk haplotypes at the IL12RB2 locus; and
treating the Crohn's Disease.

22. The method of claim 21, wherein one of said one or more risk haplotypes at the IL12RB2 locus comprises SEQ. ID. NO.: 24, SEQ. ID. NO.: 25 and SEQ. ID. NO.: 26.


SEQUENCE LISTING (38 sequences)

SEQ ID NO: 1 - DNA, 1165 bp, Homo sapiens
ttgaaatgatacttaatagccctctctattcccctgttctaaactagatctttcctctttatcttttccatcctcttccacagtgcatgactccctttcccctcatcaaacctgtcattaggtccagtcagttctacactcaaaatccttcatgggttgggcgaggtggctcatgcctataatcccagcactttgggaggccaagacgggcagagtgcttgagttcaagagttggagactagcctgggtatcatgggaaaaccccatgtctacacaaaatacaaaaattaattgggcatggtagtacgtgcctgtggtcccggctactcaggaggctggggtgggaggatcactcgagcccaggaggtcaaggttgcagtgagccaagatggccccactgcacgccagcctgggtgacagagccagaccctgtctcaaaatccttcatatccacccccattggcttccacctatttcagactaataatccaattctggattattgtatagccttttgatttttccactaccaccactttcatttcccacaaatcctccgtaaaggtgctagacagtcccagagaccctcaaaatagtgtgaatctcactatatgcactcactttctatcacttacatgatacagtcctgatccatcagtagctcatggaaggtcttctgacaggcctgctccactacaagtttcttcttgcattttcaggatccaggctattgcacttgttgaaccttctcagaaatgtcatgttatttcaygtatttgtttcagaacatgctggttgtctctgtccaggaggttggccttccccctaccccattgcgatctctcaccccatcctgactcctccccaccactccatcctcaccctatgtcctgatagaaatgtctgacttcacagtccttgcactgaaccaaaatgcagtgctcttttccaaaggcagctattctggctattccaagctatgtccagtgccgacttccctaacaggcacagtaggcacagtggctagggcccacaataattttaggagtccatgaaaatgtttaattttacttaaaatcagaagagaaaaataactgttatgttcatgtatatgcatgtatatggcatgcatatgtatacatctatgtatatgtgtgtatatttt

SEQ ID NO: 2 - DNA, 692 bp, Homo sapiens
ctggggctctgaaattgcttaggaccattttaagcaccctcaaggccataaatttctcacctcctcctgtcacccacctccacctctgagttcggcttggccactgttatagcagcacaagcattctaggacccttttggcaaaagaattattctgaggagaaagtaaaaatctgtttagtcttatgagaaatgcagatagcayagtaagaatcacagcataaagcaggtcagtgcaatccagatttaagtctttaagtttgaatgagttcatatttttgcaaactggcatttattatgtaatacatacttgaatatttagtttgttacacaagactcagatgttgaatttttattcttactgattaggtttcacatatttccaccagatcttacattttaaaaaagtatcgggaggtcgaggcgggccgatcatgaggtcaggagatcgagaccatcctggctaacatggtgaaaccccatctctactaaaaatacaaaaaattagcggggcgaggtggctggcgcctgtagtcccagctactcgggaggctgaggcaggagaatggcgtgaacccggaaggcggagcttgcagtgagccgagatcgcaccactgcactccagcctgggcgacagagcgagactccgtctcaaaaaaagaaaaaaaaaagtatcaattttattatagatgt

SEQ ID NO: 3 - DNA, 705 bp, Homo sapiens
ttgtttgcatacacttaaatgggatccacgttctgcatcatttgattgataatcaagtgaagatcctgctgaattccttttgcatatgcagaatttagattaaatttcaaaacaacacaaatacaattctcaagtcytagattctgaattaatggggttttatcctaataagacacctggggtccttgtatagtatcacagtcatagaatgatattaaagaatactgagtttcttaggctgggtgcagtggctcatgcctgtaatcccagcactttgggaggccaaggcaggcggatcacctgagctcagggattgaagaccagactggccatcatggcaaaaccccgtctctactgaaaatacaaaaaatttagccaagcctggtggtgtgtgcctgtaatcccagctactcagaaggctgaggcaagagaatcgcttgaatctgggaggtggaggttgcaatgagccaagatggagccactgcactccagcctgggtgacagagtgactctgtctccagaggaaaaaaaaaaaaaggataccaaatcctcttacttcatgcaaataggagtatgtaatagactagaaaaagtgtttagaaaatagaaaggaattatattatcagtgtctctgaataagttttcagaagccaactgttttctggttgaaactcttattctctgctccccctggtggtgctacat

SEQ ID NO: 4 - DNA, 1077 bp, Homo sapiens
cagctaccatttctccaccccattaaaagagtatattccaaaattaagaatatattccaaaattaagaatatattccaaaattaaggctgggtatggtggctcactcctgtaatctcaacactttgggaggccaaggcagagagatgacttgtgcccaggagaccagcctgggcaatataatgagaacttatctctacagaaaaatttaaaaattatccaatcatggtagtgcatgcctgtagtcccagctacttgggaggctgaggcaggaggatcacttcagcccaggaggaggtggaggttgcagtgagctgtgatcgagccactgcactccacagtccagcctgggcaacagagtgggaccctatctagaaaaaaaataaaataaaaaatatatatatacacacacacacatataaataaataaatatatatacacacataaataaatatatatacacatatatataatatcacatttggactttctggagatttgagacagttgtcaaacataaagcagtatgggctgggcacggtggctcacacctgtaatcccagcactttgggaggccaaggtgggcggatcacttgaggtcaaaaattcaagaccagcctggccaacatgatgataccccatctttactaaaaatacaaaaaagtagccaggtgttgtagtgcatgactgtaatcccagttacttgggaggctgaggcagaagaatcgcttgaacccgggaggcggaggttgcagtgaactgagatcgagccaccgcactccagcctgggcaatagagcgagactccatctcaaaaaaagcagtgtgtgtttcagttttaatgtatttcagagacagtatttgattatgtacggccaygttttatataaagaacactttgttttcctagagtctagaagacagcttggaacataataggtgttccatacatttctgctaaataaaatagttgttttaaaagcacaccacattttattattgttacccatccattttaggttaaagaatttgacaccaattttacatatgtgcaacagtcagaattctacttggagcca

SEQ ID NO: 5 - DNA, 997 bp, Homo sapiens
catttctgctaaataaaatagttgttttaaaagcacaccacattttattattgttacccatccattttaggttaaagaatttgacaccaattttacatatgtgcaacagtcagaattctacttggagccaaacattaagtacgtatttcaagtgagatgtcaagaaacaggcaaaaggtactggcagccttggagttcacygttttttcataaaacacctgaaacaggtgagtgtacttatatattttattctgttgggcttttctttatatatcttttctgctgagcacagtggctcacacctataattccagcactttgagaggccaaggcaggaagattgcttgagcctaggagtttgagactggcctgggcaacatagtgagaccctagtctgtacagaaaaataataattattattagcctgggtggtagaatgcatttgtagtcgcagctacttgggaagctgaggtagtaggattgcgtgagcccgggagtttgatgctgcagtgagctatgatcatcccactgctctctagcctggaggaaagaccaagaccctgtttcctaaaaagtttaaaacagccaggtgcagtggcttatgtctgtaatcccagcactttgggaggccaaggtgggtggattaccttaggtcaggacttcaagacctcctcggccgacatggtgaaaccctgtctctactaaaaatacgaaaattagctgggcatggtggcaggtgcctgtaatctcagctactcggaaggctgaggcaggaaaattgcttgaacccaagaagtggaggttgcagtgaactgagattgtaccaccgcactccagcctggccaagagagagagacttggtctcaaaaaaaaataaaaataaaaataataataataaataagttaaaaacaaaataaagctacaagatattttttttctctttacctttgaccaaaattgacaaaactattctagggcagatgataacatttaaat

SEQ ID NO: 6 - DNA, 720 bp, Homo sapiens
ccagtgtgaaaatactgtgcattttccccaccatccctcagcaatttcattctttaatttcagggaagcagaggagcaacttacttaagtattctaagtataggactacaaatgttcttctttaaacataaaagtcttggcgaggtgtggtggctcatgcctgtaaccccagcactttaagaggccaaggcgagtggatcacctaaggtcaggagtttaagaccaccctggccaacatggtgaaaccccgtctctactaaaaatacaaaaattaactgggtgtggtggcaggtgcctgtaatcccagctactagggaggctgaggcaggagaatctcttgaacttgagaggcggaggttgcagtgagccaagatcctgccactgcactccagcctgggtgacagagcgagactctgtctctaaataaataaataaataaagtaaaataaagataaaagtcttaagcttcaggtagaaggaaataggaacaccacagtttaaatttaaggtctgtttcctraggagaaaaatcacttaagagacaaaaataccaattaaaattaagtatccctgaaaacttggatttattaaagtttaacatgttagctaagagaaaccatagactgttctcttggtacaaattcccttctaagacacattacatgagaaacagtaaaagtgtgttagggaaagtgctcatgttaaatctctttgaaaatg

SEQ ID NO: 7 - DNA, 1001 bp, Homo sapiens
cccatacacatgttggtaatcagaggtcacagaagtgacctgtgttgtgaaagtactatatagcaagagaaattgagtatgttctttctactcagttaccttataaggcaaaagggaattgagaggaagtggctatcctagattacatgggtggatctggtaaaatcacaagagttcttataagcagaagggagaaggttgagagtcagagaaagagattggaagatgctatgcttctggctttgaaaatgaaggatggagccatgagctgaggaatgtaggcagcctctagaatatagaaaaagcaatgaaactgattctgtcctgtagcctccagaaggaacataaccctattgacaccctgatttcagcccagtggttgtgattttggatttctcacctccagaactataagataataaattcatgttgttttaagctttcaagtttgtgatgatttgtgacagtagtaataggaaactaatatagaagatgatgacytcaagaaaaagcataatcataggccaggcatggtggctcctgcctgtaagcccagcactttgggaggccaaggtgggcagagttcttgagtccaggagttcaagaccaacttggcaaacatggtgaaaccctgtctctacaaaaaaaaaaaaaaaggaagaaaaaaaattagctgggtatggtggtgcatgcctgtagttccaggtacttgaaaggccaaggtgagaggattgtttgagcccagatcttatgagctgagatcacaccactgcactccagcctgggtgacagagagagaccctgtctaaaaaagaagggaggaaggaaggaaggaaggaaggaaggaaggaaggaaggaaggaaggaaaaagaaagacagaaagaaagaaggaaagaaagaaagaaagagagagaaagaaagaaagaaagaaagaaaagaaagaaagagagagacagagaaagaaagaaagaaagagaaagaaagaaaagaaagaaaggaa

SEQ ID NO: 8 - DNA, 701 bp, Homo sapiens
aggtgcggtgcctcacacctgtaatcccagcattttgggaggctgaagcaggtggatcacctgaggtcaggagttcgagaccagtctgaccaatatggtgaaatcctgtctctactaaaaattccaaaaaaaaaaaaaaaaaaaaaaaagccacgcgtggtggcatgctcctgtaatcccagctacttgggaggttgagacaggagaattgctagaacccaggaggcagaagttgcagtgagccaggatcatgccactgcactccagcctgggcaacagagggagattctgtcttaaaaaaaaaaatccggttttgattatgtcttcatagcagtgtgaaaacagactagtacggttgatgtagaaagaagagctgaggtgatgatttggcatcatccttaaaatacagatggaatacgttattgctaaaaccaggtccttttgagtggatttgattaaactagcctggtgttttggtaggccaaaaaatatagttgttaygctttaaattttgtccaacaataagaaaccatatttctcgtttgagatcactctaaattcccacaggcacattgtcttcttgtaagactaaagtttggtgccagtgtgtacaagttatataaaaattcttcccaaattaaagataatttggattttttttagtatattcaagtatgtcctgtgagattaataggcataag

SEQ ID NO: 9 - DNA, 886 bp, Homo sapiens
tatttgaagcaactaattgggggtactggctgccacacacccttgggcattaattagtgcctggaagaggatagacagccctcaggtcaacacagtgctcggcaaaggggtctaagcagtagagcagaatgaccaagagcgtggcctgatatacctgggtttgaattaaactctgcctcttatcagctctgtgaccttggggcataattatgaacttgctgagtctcaggttttctcttttggaaaatagagataataatacttatctaacagagctgccatgagttcctaacctccactgatcccacagaaatatcaaggtgtaggtaggtctgtgtaggcatctataattagggaactgtactgaacctaagcacttggcttgyaattgattgataattcagagtgcccttacctttcttcatgtttctttttctttttcttctttttcctcttttttttttttttcctgagacagggtcttgctctgttgcccaggcgggaatgcagtggagctcactgcagcctctatctctggtgctcagttgatcctcccacctcagcctcccaagtagctgggactacaggtacatgacaccacacccatctaatttttgtattttttgtaaaaatggggttttgccatgttgtgcaggctggtctcaaactcctggactcaagcaatctgcctgccttggcctcccaaagtgctgggattacaaaatgtgagccaccatgcctacccacttcatgtttctttacgacacttcaccaccacctgacttttcttcttgttttgtttgctgtttttctgccctgtctggctagaatagaagctccatgaagacaggggctttgctcattgttttcactgctgattccccag

SEQ ID NO: 10 - DNA, 601 bp, Homo sapiens
ttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccraactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagcc

SEQ ID NO: 11 - DNA, 601 bp, Homo sapiens
tttgaacattaaaatatttcaagggacttcataatcaagtatattttaaaacagcctcaaataaaattccgtattagtttgccttccttacaagggtattaggaatatgtttattaatgtgtaatttaaattttgaaatattaagttctgagcaaaaaacctatgtagataagaaatcattagtagactttataatagctcatttaaaatctttctactgcacttgattataaatgtaaacgaaagaaagattatttcatgaagcaaatgatggcaagaaggagaaactcagtgccaattyggcaaagaacattcaagtcaaaatttgtgagcaactggacacactggggaactgccacaccaaacaactctaatctatcgagcagcttagaaatactcaatgcatcagtaaaatttagaaatccaagggtcttgcttttctcaaagtctcattttaaataactaaccatagatctttactaataccatcacaggagggaaaaaactgaagggggccaagagtaagggactttggggctgaatgctaaaacactaaaacaattggtaaggaattgacaaatttaaaaattgtcacacttcc

SEQ ID NO: 12 - DNA, 601 bp, Homo sapiens
ttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaagragagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacgtgcgatgctcttgctgatttggaccagatagtatttctggaccgtgggcatgaaacgct

SEQ ID NO: 13 - DNA, 801 bp, Homo sapiens
aatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacrtgcgatgctcttgctgatttggaccagatagtatttctggaccgtgggcatgaaacgctgggttctgactatggagatccaggaatactgtatatgtaggataggaaatgaaagctttggtaggtatttaagtcattgtgcagcattttcaagaactgatacacagcagtttgaaagataagattaaaactgaaagatagctatattggggctaaaccacacaagaagtgtcacatgatgctgtgcagtaagaaagaaaatttattgaaagtctgtttttctgagtacaaaggatttaatataattctcccacggcatttttctttaaaatgggtcactatccttgagattttgaaagccgtagcagcaacaacctttgtttccattatctcgtaccata

SEQ ID NO: 14 - DNA, 511 bp, Homo sapiens
ccatggctttaaaatttttttaaaaaaactagtttcaacattctccttttgacttaggaaagacatgttatccattggttggcaataattttaataaaaatgtcaagtcatggcatgtcattagcctatcagcacatgcatcattgtcaggtctgggaaggaataataaccttgattttctaggtagaaatatcctcctgcaccattgttctcagtcccatattctgtgaaactcatcgtgaagtcaaacattcamattggaagaaagagctatagaaaatctatgtggtatcaatattcatgctagaagtgctgttggtgctactggcaggcatccaactaaaaactcgatctccttcatgttttcttaggtatattttccagttgttctaaatttaacatgtattgattctgtaataaaatcagatttcaaaaaagatacttgaagttaaatatttaaaaaatataaaccccacttattctaaaacacagttatacctatgtttagtta

SEQ ID NO: 15 - DNA, 1293 bp, Homo sapiens
gaacctgggtagtatggtattggtggggaggtgggggttccttggagaacttttggaagtgagaatatagtatttggtgatatgtggatgttaggaatgagggagaggcagaaggaaaaagattcagggaagccacatagatttctagcttggatgactaggtacatggtagtgctaactggggaaaatgaagagagaataaaagcaaagtgtatcaggggaggagtacatggaaagcaactgcctcttcccatccgcataccccccacccaaaatctagtgggaaataatggttcaggaccacacacacacacacacacacacatatagacatatacatcctttacaactccctctcccaacaaaaacaaaaacaattttttcttttcatcatcaccgttcagagaaagcttgaaaacgagcagcaggtttttagtgagaagcttgaaagcgtaaaggctgtgaggaactgtccctggaagctgcctggggatttcctgtaggaaaatggtgacagggatggtcacaggaatcaagatgtgagcacaaaatgactgagaggaggtggctggagaggccaacccctggatttggaatagggaaagaagcctagaaaagccatgggcctctgggtgggctggagcacactggatggagcaggatggagtgaagaggaaggtctttcaagaagcagggagcctgcagagtggcctgagaatatctagaggccttcagaagtagggcaagacagcacatgggccatgggggcgaaaatggttacgatgtgaaacttgaaactactctggaattgaatgtgattgagtttttattttacttgggctgaacttttctcatacttaaagttcrttctgccccatcagctcctttctgggttgtgtggtgccttgatcagacagaagccaggccctaggagtgttgcttgaggaagagaaaaatgttggtctgttgatctctgaggggccttaatctccaaaggaagcctgagtctaggggagaaactggacattgtagtctgaagacaatgtctcctcccagaacttcttgtattttggggagggtttcattttccccatatgatctttaataatgacatgccattcctcagggccattatcttatttgctctattcctatcaaaatgcttctgtctacagcattggctaatagtatgaaaaccttagtcggtgttcagtcttgaaggcatgtgaaatcgagaacttggaattttgggtatttccaggtcattgttactcaaagacatgctttgttttg

SEQ ID NO: 16 - DNA, 801 bp, Homo sapiens
gcactgcccctgcctttctcctgtctggttctcccaccctcacctgggcaggggttcgctgacccgcccttgctggagggagatgatggtcacctggagatcgtggtgtagccagccaggatcccctcctctcacattgccgctgctggctggaaggcatgggcgctctacagttctggagcccttttcctgccctctctgcccgcagatccagcccttcttcagcagctgcctcaatgactgcctcagacactccgcgactgtttcctgcccagaaatgccagacactccaggtaggggacatgcggctgtcctaggccatactgggagaacaagtggctgaaggcccccagcctgtgctgcgtccttacctggttctgaggggtgattagggaggagastttagtttaacttggagtccttcaggcctgaagtgtggagtggggctttagagtgtcactccctggggctggactcctggctgtctttcattagctatgtagccttaggcaaattacttaatctttttgattctcaacttccttgactggaaaatgaggtggtttttatcctagagccctagttctgtgccatgcactgagcgcagtgctccaacatgccgtccattttttcatcctcactcattgtgagtcacggtactatgcagtagaggatccccccaccccaaaccccaggttcctggataaggaaactgaggcacagagatgttgaataacttgtccaagatcacacagcagggacgctgttttcaaaagtcgcatgccctaatgcacgggaggc

SEQ ID NO: 17 - DNA, 801 bp, Homo sapiens
gcagtagaggatccccccaccccaaaccccaggttcctggataaggaaactgaggcacagagatgttgaataacttgtccaagatcacacagcagggacgctgttttcaaaagtcgcatgccctaatgcacgggaggctgcagccacgtgctcaccagaaggcaaggcgcaggcatggagccaggctggaaggagaacccagcctcccaaggaggaggcaaggtgtctcttcttagaccagcaactcaagtgtctcttgtagatggtttcattaagttcaacctggatctagagtgcctggtgcagggccaacatcattaaagccctcaagggacgtcagttgtgtttcttgtgatgactgggaagggttaagaatgctattttccctttttcctctgttytcattgcagaaccaattccgggtaagcttggatctctctccgacagcactgcagccctcaggggacattccccagtggccacttgagaagtccctgcctcagccaggcagacaaggctgaaccgaggccagcccggggtggggggtgagaccatggtttgtcgtggtggggccagagaggacagagcctggggctggggagcagggctgggggcctcagggtgggcagggcaggccccgccgcatcactcacgctgttctgctcaccgcagactacatgcccctgtgggtgtactggttcatcacgggcatctccatcctgctggtgggctccgtcatcctgctcatcgtctgcatgacctggaggctagctggtaagcgctggggctctggctgtcctg

SEQ ID NO: 18 - DNA, 801 bp, Homo sapiens
ccagccccacctcattagccttgtagtcacaggccagttacttaatacaccatggattcacttttctgtaaaatgtactgataatgcctccctctaagggtgtgacgaaggttaaatgagtagctgaggaaggtgcttgctggtggggattagtacataccagtgtcttctcccacctgcagccctctgctggccaagtcctaagccgggagaacacaggccttccggttggggcttcagcccttgcctgccccaccatgaccctaggctgctccttccgtcatctgggaagctgtttccacccttccctaggctcgtcaggattaggtgttaatcattattaattattatgtggtagaaagaaaaccagccaggcatgggaggacctatgggaggttccrataacattcagtagcatctcggccagtgctccacaggcggtgcagctctctaaaggtttggggctgggcggcggcggcgcttttggtttcctttctgctgttgcgcttctgttttccgaagtgtcctgcaccacagggtgaaggcaagaggagcctcgctgttatttggctgtcttgtgacagttctggggaagagctgaaagggttaggattgagattaaggttctaagtcgtttgctcagtcatctgtggatctcaatcctcccagctgtcactaaggagttaacccccgcagagcagttttttcatcacatctctgaggggaacaattgcttaagtatgtgggttccccttcctcacctcaaaaataccaggaggaaatgttgcaagcagcctggtg

SEQ ID NO: 19 - DNA, 768 bp, Homo sapiens
tcagtgctacaaaataactgtgatcccaattgatwatgtacaacgtgccaggcacgtcacatacacacactcatttaatacccattaaacaagagcaaatacagacccacctcacagaggaagaagctgcatttcagaggcactagtaactgctccaggtcatagtgctcgtagtggcagacccaggactcatgcctgtgcgaccacctagcacggcctcgctgctcagtctcggggctgcccccttacccttcaccctttgtcagggatggggcagacaccctgtgagctggtttctatttctcttcccaaagaaccactccagtgtatttcttttcctttccagggcctggaagtgaaaaatacagtgatgacaccaaatacaccggtcagtatttcctggtttgcatgtttgcttatttttaaagcagtggagggttctcctgggataagtgcgtgggtcgcctcctgtgctctaactcccaagtcccttcaggagaccccaccttagaaacccccttccagtaccccactcagaagggcccccaataacaaggcctggtgccattttttgaatcactcagacaaggaaagaaaggtaagtattttgtgaacagaggtcctccctggagcagaccaacagagcttggtgcctctttttcttttcttattaaagataggctaagacagctgggacgttgagagatcttttccacaggcagcagactgattttttcaggcagcaagggtggatagtacatctgggtt

SEQ ID NO: 20 - DNA, 829 bp, Homo sapiens
acctcgctgaagtagcagactacgtaggtgccgaagcaggctggcctcttgaagtccgggaggatcatgttcatggctgcagtgaacaggtcccccacgggctttccgtggtcgcagcgcagccgcacaggcgccccccggcccaggagcgcctgccacttggcgcgcgtgccgcgggagcacaggacgatgatcttagagttgctctccaccatctcctgcttctgacggcccacccaggtcatgactcctgcctccgagatggcctgctcttccagcaggtccagggccacttccgtgccgcaggcggtgagcaggaactgggcgaatttcaggaccacgtccacgtagagggggtggtcggctgagtagatgatccagactttcctgggcttcagcggtggggggatcaggtcagccrcaggcaggccatctaaggaaacaagaccacacatgctgaccctcaccccagggcccagggcagctctgtgcctgccagcccaggaggggcctggaccaggacacagagcttggctccctccctaagctgagaaacccaactgaggcctgttggaaaaacccagatgttactattccacctttggctgcctgaaaaaaatcagtctgctgccttgtgggaaaaagaatctctcaacgttcctagctggtctttagcctatactttaattaagaaaagaaaaagagcaccaagctctgttggtctgctccagggaggacctctgttcacaaatacttacctttctttccttgtctgagtgattcaaaaatggcacccagcttgttattggggcccttctgagtgggtact

SEQ ID NO: 21 - DNA, 852 bp, Homo sapiens
tgtttagccctcagcctctctccatgcagaggctcatcagacgaaaggtgccccaggcctcaggactgatgcgcacaaggctgtccccacccctgagctctggcgacatccccccaacccccaccccgatctctctcactgcctccctccttcccctccaggctccaccagcagctccctgacaagctcactccactcacctcccagcacttacccacaaactgcttccttgctgggactacgctttccccaaccacaatccttcasctcaggcatctcctcggggatcccccctgacctgggtgcctttcccgtgcatgctcacaaccctgggcaggcttccactccatctttctacttttttattttttttgagacagggtctcactctgctgcccaggctggattgcaatggtaccattatagctcactgcagcctctacctcctgggctcaagtgatcatctggcatcagcctcccgagtagctgggactacaggcatgtgccaccatgactgactaaaaaaaaatttaggtagagatgaggtctcactatgtcgcccaggctggtcttgaactcctgagctcaagcaatccacctgcctcgccttcccaaagtgctgggattacaagcatgagccactgcacctggcccattcagcgtttacatcccgcgtgaccatcttttttttttttttttttgagaagagtctcgctctgtcatccaggctgcagtgcaatggcacaatctcggctcactgcaacctctgcctcccagatcaacattctcctgcctcagcctcccagtagctaggactcagcatgtgttaccatgccccggctattttctatt

SEQ ID NO: 22 - DNA, 511 bp, Homo sapiens
acctcaccctgtccctacctctgtatgacattgagtaagcagcaacacctctctgggccttagacacccgctgtgtgccttccctgcaggtttgaacccaccaggacctaaaagggagggcacagaggaggggtaggcgcaggccattccaggccattacccattccagccggatccgggtcttgcggcgcagtcaggctgcaggtggcaaggcccccgtcctggcccacaggctgccattcaatgcaatacgtcrtgctctgagcccgggctggccaatacatggtggtcccgttggttccgacgctgatattcagagccactggttctggaaggagaggggagagacgcatcgagacagttgccatctctctccccagtccccttctctggctcctgcagcctctctcccaccctctcgtctatacccctgaccctacagggccaggggtgtctgtgtttggctgtttccctgcactggaggctgccttggggccaggcacaaagtggg

SEQ ID NO: 23 - DNA, 643 bp, Homo sapiens
  misc_feature (18)..(18): n is a, c, g, or t
ggacacagagtgagaccntacctcgctatgttttacataaataaataaataaataaataagtaaagtatacggaaggatgtgcataggttatatgcaaatactttaccattttatatcaggaacttgagcatctttggattttggtatctgcagagggtcctggaaccattgccccgtaaatatggaaatgactgtatacattatattatgtatgtctatctattgtaaactacatacataagatactatactatgtatacctattagatacatacttgctttttattccttccttagttttttttctcattgaactccgggaggtggaaaaaatttagtgcatttcacagaagagaaaatctagaccagaggaaagatgtctaagttcgatcccagaagccatgactgcccatctcccatttgacagcaggaaagactgaggcacagagagatgaaaccaacccacacagcaggcccacagctctccacacatacacgtgcctccacccagcaagaggagccgccatgccagggtcaggggactcaccgccctgcctggacagcaggaagaggaagaggagggggaccacccaggtcaccagcggctccatcrgatccacgtagagccccacagccccaggg

SEQ ID NO: 24 - DNA, 601 bp, Homo sapiens
ggctttaagaaagaacttttttaaaagctggattcacttctgaatctttaaaataatcccccttaatcaaatctcattttcaccaaataactactaccaacattagtggtaagtttcaactacacttagtccagtggtttttcaaaaaccaatgtgccaaacccaagagttccaatgactgagaagccagagacggctagtgatgactgcctcaggaggagaagaaatgttcgtaggaaaattcatagcaaagggacagaggaaaagaacaagggggcgagaaggtagcctagagatgaayggtctatgaaagaaatgggaggaagcctgagaagaaatatggtggaaaagtgtatagtaaatgccaatgatcagactcatcgttagcaatatgtatgcagagaagtgccatggaaagagaggcgagaagcacatagtgtgaggttggaacttcaggcagcccagttctgcctggatggtcagaacttggaatgcttgagattggcctctgattcaactggaccgattacatttgctcagaaatgtactggctcactccagggatggaggtgagtattctcagagcaagatgcatctgtga

SEQ ID NO: 25 - DNA, 601 bp, Homo sapiens
accttatccagaactgctgcatcgtgactcatctttgttgaaggaaaatgctgtgcgattcgaattgagctactgattggcatgctcataagaggtttcctggtgaactgcactctttatttctcaggactcacatcctcttcactacacctcttccagttgaggatagaaagttttggaattatgaatctgaatgagcacaaaaccagaagagtaagaataacaattataaagtcctctgatggggttcacatgatgggttttgatcattgcacacttcttcagttttaagaaatgagtkatttcttcttctttctcctccctgtctctctcacacacacataaaacgtctggtataagaagagactactataaatagcctggtgaatcatgatcaggctcttcactttgaaactaacactgcagctcaaaatttattttaaaatgaatctactttcaatagcaagacaattctcaaagcaggacatttagaacagccataatttgtgaatattatagaagaacccagcctaattatttccttttcacgagcaagtgactggttaaaaccaaaattcctcctctcatgtaaaggtttctc

SEQ ID NO: 26 - DNA, 644 bp, Homo sapiens
aagatcagttacagtgcaggctaaactgccttaaaaaaaaaaagaggacccaaagcacagtggcttaaataatactttatttatcttccatataacagccctgaggtaaggtatccagactggtgggcagctgtgctccatagttatttgtggactcagttttcttccatctttttggcataccatcccttaggtattgtcctcatctgcatgatctctatgagatcacagacacttctgctcatatttctttggagaaaacctagtcacatggccacatctcttacttcaaaaggttagacaaattgtaaagcatacttttaaaaaaaaaaaaaaatcaaagcttatgaaagccaagtctttaaaaatgtttcatattgttcatgaactttaaaaaaaataagtcacatgcatttataagaaatgtttttaggatttttcctkctacatcataaaagacctgtatttcctcttattgccaaatacaatcactgttaatagatatttcagatatttccctataatacttttttcttggcatttttaacatggttgtgttcaagctatttataccattttgaatcccgtctttttttcaactgttcacataatttacctgtcattacaaactctttgtaaatatt

SEQ ID NO: 27 - DNA, 2826 bp, Homo sapiens
acaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 28 - DNA, 2800 bp, Homo sapiens
ttttttttttttttccagacagagttttgctcttgttgcccaggctggagtgcagtggcacaatctcagctcagtgcaacctctgccttcctggttcaagtgattctcctgcctcagcctcccaagtagctgggattacaagcacctgccaccatggccggctaatttttgtaattttagtagagacagggtttcagcatattggccaggctggactcgaactcctgacctcaggtgatccacctacctcagcctaccaaagtgctgggattacaagcatgagccaccacgcctggcctaatgattctattttaaaggaaaattttcagcatttattttactaccaggtattttgcattatttaccctttctgtacacaatagagagctgttttgcatctttcaatgagggaaaataaagaggaaatagttgactctataaaaaatacatgaggcgtccacataatgctgtttttgtgcctgtatgtgtgaccatgaagcatgttccaccttcgggaccttaattctctaattttaagaaatgaggcagaagaatggctaaaattaaaaaggcatggagctcccgggaattcttacatggtgatggtaggagcacagaaatgtcactactctggaaacaagattggtagaactgaaacatatgcttatcatatggcccagcaatcccctacctaggtatttatccaagggaaatgaaaacatgtgtccatgtgaagatttgtatgtgaatacatttttttcaaaaagaggcagaattcaatttctattgcagggaaaacccaagttcagattgcaaggcagctttctcatattgattttgaccacacagctctacttttccaagagtgaaatcctattgaagtggctttccaaaatattttgtggaaaataagtattaatagatggcaactcctcattcacgatccccaaacaggagacaaattcatcaggaagcagggtctgttctggaatagtttcactgggtgaatcattttccaaaagcatggtggtttcctcctctactgagttttgtatgtcaggagaactgcattctccttgatttaatatgaggcttaattctccaagaaatattgtgttgcttagtgaattcacacttgaaacagaaaaagcaaaattaggatgcttttgtaacctgggattatttcctgagtctaaggaatcaactggtggtttaagtgttaaggaagtaatttcattattattgctgagatggcttccctcaggcagaaaatttgaaatttggggtttatatccagtgttgagatcaggaatatatacaactgtagtattgtcgaatagcgagttttgcgggtagtctcttgtctccaggggtcctgtattctccttcttgtagtctgtaggcttgtgttctgggatgaagatttcttttatctctgtaatcatgggatcaacatataggacctgctcactggaattattattcataagttcactattttcctgtagcattttcacaacattgctgtttttcatattaggaatatcttcataaagccactttggtattaacaataagatccttcttttaatcccagttcggaatgatctgttaaatatcccaatcaaagaaagaattgacaacataacagcaaagacgatcattcccaataaaagtccaatgtctcctctgttgtcagaagtaaggtgccctgtagagatggaagcaactgttagcccagaattccatgtgtcatgttggaatgcttttgatgtgacctggggaactgtttcaggtgttttatgaaaaaacagtgaactccaaggctgccagtaccttttgcctgtttcttgacatctcacttgaaatacgtacttaatgtttggctccaagtagaattctgactgttgcacatatgtaaaattggtgtcaaattctttaacattccaagtttggtttgttgtagccttgtatctcatttcacaggaaaccttttcaattgttgtttgactatcccaataaattatggtcttgggcactgtagcatttatagtctcagccctggaaatgacggctgcagaaggtatcactatatcatccaggtgaatttgcagttgttttgactcttccatgcctagtgcgtttgctgcttggacccaaaccaagtacttcttgccaccttgtaatgaatcagtggagatgttaatatagcttgaggtgagatactgttgctcttcttctgtctctaaactcttcacatgtaccacgtattttgtgtctatgtaggtgagcttcccagcattccaggtgcaagtcatgttgcctgaatattcataaatgacacaggttacttcatcaggaatatctggcggatatccagaagaaatgtcttttccacatatcagtgtctcttgaaaatgtttgggacattcagcagtgcagtacatagaagcatgtggttccagaaagtttttataccaaagccgagctgttgttttattaatccttgtgatttgaaatctttctttgatgccatttttataaaaatgaagtttccttggttggcagttcttaattgctgcttggcaatatatagagatattcatacccatcttaaaaattgtggctggttctacccagatgtggccagagcagtttatatttgtaattcctccatgacaccagctgaagagtatgtaaagggctattactgcatcccattgaatagtgacctgattcatgtctggaagcaggaaaagactgtttccctctttcaacctgtttgaagcacataattcca

SEQ ID NO: 29 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 30 - DNA, 1859 bp, Homo sapiens
gggcgaaaatggttacgatgtgaaacttgaaactactctggaattgaatgtgattgagtttttattttacttgggctgaacttttctcatacttaaagttcgttctgccccatcagctcctttctgggttgtgtggtgccttgatcagacagaagccaggccctaggagtgttgcttgaggaagagaaaaatgttggtctgttgatctctgaggggccttaatctccaaaggaagcctgagtctaggggagaaactggacattgtagtctgaagacaatgtctcctcccagaacttcttgtattttggggagggtttcattttccccatatgatctttaataatgacatgccattcctcagggccattatcttatttgctctattcctatcaaaatgcttctgtctacagcattggctaatagtatgaaaaccttagtcggtgttcagtcttgaaggcatgtgaaatcgagaacttggaattttgggtatttccaggtcattgttactcaaagacatgctttgttttgcttataggagaatttgcataactcttcccaatagggaagagctgagtttgctaaaatacatttaatgagaacaaataaacattttcttttgaaactttattattacagaaggattttattataagggtttaatggaacagagagttagacttgctgtatctcttgtaaaacctcaacaaattgcaaaatttaaattaatacatatgaaagtaggtttaatctttcttttcagccatgttataacttttaaaaacaaagaggaggaaatactatatccgtatgttgtaaagactcagaaattttgcactgccatttaattctatcttacatttacctaggcagaactgataatagtgctttttcacctttttctaaatacttaaacataaatagatccttaaatagctcaaataaatacaggttttaccttttaaataataaaattcccaagcccagaatcttatcttctttaaaaagacaaacaaaataaagtaggtaattcattaaaatggaatcttataccccaaagttatctcagggcatattaaatacataaataacttaaagcccttaaaaaagtaaacaagaagcaaattggtactcagtcaaaccttccttcttggaaagagggaaaggtaattcagatctcatatcttggccaagtgttacctctgaagctttaattggacaaactgccttaagaactctaatgagtttagtccgaaatgaggctgtctttgaaggatgagggttcctgaggggctgggtcggctctccatagtctaactgctttggggagtgtgggctccccagagctcttaggccacatggtggacaatcggggtgacacaggtgcagcccacggacaccagtatcttctccagccggaaggagttggggcagtgtggaggctccctgcgcaggaccaggatctcttgctggatggggacagagttcatgtggtagtccacgttcccatcagcgttgatgcagcccaagtggcggcactttgcctcccagatcacagagggatatctctcagggtcctcattgcggtggagattccaaggtgaggtggatcggttgtagtaatctgaggaccttttgggattggtattggtattccggttatggatgttcaggttgaccatcacagtccgggggaagttcttgtcctcagaatttgggcatcctggatttcgtgggattgtgattcctgccttcactatggcctccaggctcagcagcagtagcagtgacaccaatgaggtcttcccaggagtcatcgttgtttcttccaatcaactggggatggatgagtttgtgcctgc

SEQ ID NO: 31 - DNA, 3429 bp, Homo sapiens
ctgggcccgggctggaagccggaagcgagcaaagtggagccgactcgaactccaccgcggaaaagaaagcctcagaacgttcgttcgctgcgtccccagccggggccgagccctccgcgacgccagccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 32 - DNA, 3429 bp, Homo sapiens
ttttttttttttttttttgagatagagtctcgctctgtcatccaggctgcagtgcaatggcacaatctcggctcactgcaacctctgcctcccagattcaagcaattctcctgcctcagcctcccaagtagctaggactacaggcatgtgtcaccatgcccggctaatttttctatttttagtggagatggggtttcaccatgttggccaggctggctcaaactcctgacctcagagtgatccacctgcctcggcctcccagtgtgctgggattacaggcgtgagccaccgtgcccggcccatcctgttcgtttaacgtgtcctcggcactagcggttaagttccttcatggctggggccatgccgcatgcaatttatcttggtatcccaaatgcccgccacatagtaggtgcacaataaatgctgaatgaacgaataatggagcctcaaggacgctctaccattagctcctgggagcgggctgtgtggattcccctggggatagacgataaccagaccgctgcacaaagaaaagttaggagggatgcctgggattaaaatccagacatttacattttaaagcctacatttcacacacgaacatatacacatgcagacatgtacacacagatgaatacgtgcacacacactcctctctcaaagctgggatctgggcggtccctggggagccgccctcatgcactgggcccctctgactctgaccccatcgtgtcccagcccgagttcttctgcagctggcggaaaagcagctgccgctggaggctcctcaggctctccaggtcctcgggagagagtggcagggccggcttccctgggtcctgctcctcttcctcctcttcctccatttccgtgagtccctcggggggctgcggggagctcctggagatgtagccctggtcagactgcactgactgccgctgctcctcctcgtagggcgtgtgtgggtctgtgaggaccatggcgggtctactgcagcccccctgggcctggcagctcagactctgctcgaagagcgagagcatcaagccttcgaggtgctccctcacgtcctctggaaggaggtcaggagacgccatgggggtgctgctgccaaggggcgagtcctcggggtccacggggaggaagaggacgctatttcgcccagcgcccgggctgcccagcagcgggcaggcctcgccctcccccgccagtgccagccggactgcggcaccgtcagccaggggcccaggctccaccgcggccaccagggccccctcctctgcggcgagcaccagggtgtggaggggctgcggcgctggctgaccccggggctgcaggtgaggttccagctttgccactgctgctcctccttcctccccgaccagcgggtctatggccaggcaggcctgggagccaggctcgcgcaccaggggcgcccgcttcacgatgccggttcccggaggcagcagtggctcctcaaacacctcttcgtccagggacggggcatcctggtcatctgctgagtagaggttctcacattcgaaccagtcgggacagcggacctgccagtcccggaacctgtccagggcggcgcggagctgcctgccgcccgggctccgcaggtagttgtcccccgacagctcccctacgcggtgcatgcggcccggctggaacatctccaggtcctggatgcggaagtacacctcctcgaacctgtccatgagcgggtaccgcggcgccgcgccgaacaggtcggggacgtcgccgtcacagctgacctcgctgaagtagcagactacgtaggtgccgaagcaggctggcctcttgaagtccgggaggatcatgttcatggctgcagtgaacaggtcccccacgggctttccgtggtcgcagcgcagccgcacaggcgccccccggcccaggagcgcctgccacttggcgcgcgtgccgcgggagcacaggacgatgatcttagagttgctctccaccatctcctgcttctgacggcccacccaggtcatgactcctgcctccgagatggcctgctcttccagcaggtccagggccacttccgtgccgcaggcggtgagcaggaactgggcgaatttcaggaccacgtccacgtagagggggtggtcggctgagtagatgatccagaccttcctgggcttcagcggtggggggatcaggtcagccgcaggcaggccatcggtgtatttggtgtcatcactgtatttttcacttccaggcccagctagcctccaggtcatgcagacgatgagcaggatgacggagcccaccagcaggatggagatgcccgtgatgaaccagtacacccacaggggcatgtagtccggaattggttctggagtgtctggcatttctgggcaggaaacagtcgcggagtgtctgaggcagtcattgaggcagctgctgaagaagggctggatctgcacttggtggcgacagcaccctttaaggttgcgtagagtgagtgtgacgttggatcgctggtggaactcttctggtctgggcgcaggtatgtggtgcatgtgctcaaagcaactgtggttctccatgtgcggaaaactggtcagcaggatctggtaatgggtagattcgttccacagggtgaagctcacacgcagctggtgggcctccagggtctccacggtgatgttggggtcccacaggctgcctgagctcatgcatggcgtggttaccttcatcctggcgtgctcacagtcaggcacaaggaaattcttggactggtggtttgggtccccatcagggatgggcttgggcaggtggtgaacggtcacctcatattcctggtcagggtcaaccacaaagtggctgaaggtaaaacgccaccgcctgtggtgatgcctcagtttggacagaaactcaaacctgacgcacaaacgttcattggtgttcagctgcaggacagataactctgcaccctcgaggtacaggatgctggcgtctgtctgcagtgtccattcgatgtgagccacggggaacaggtctccttgttgggtgtgggcaaagtgcagctggatctgcaggtcctttggggaggagggggtcaggtttcgagggtgaatccagctgtcatccaggcaggtactattcttgaccgtgcagtttagccccggctgggagcagaccagcgcccggtggtccaggagtcgcagggaggcgccacccggggccagcacgcccaggagcagcaggagcagccccagcaggggccccgggacagcggacggcgggctgcgtgcggcccccatggcccggctggcgtcgcggagggctcggccccggctggggacgcagcgaacgaacgttctgaggctttcttttccgcggtggagttcgagtcggctccactttgctcgcttccggcttccagcccgggcccag

SEQ ID NO: 33 - DNA, 2100 bp, Homo sapiens
ggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagagcatcccgtgcagcccacagagacccaagttaccctcagtggcctgcgggctggtgtagcctacacggtgcaggtgcgagcagacacagcgtggctgaggggtgtctggagccagccccagcgcttcagcatcgaagtgcaggtttctgattggctcatcttcttcgcctccctggggagcttcctgagcatccttctcgtgggcgtccttggctaccttggcctgaacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtgatcgttgaggctcagagagggtgagtgactcgcccgaggctacgtagc

SEQ ID NO: 34 - DNA, 2100 bp, Homo sapiens
gctacgtagcctcgggcgagtcactcaccctctctgagcctcaacgatcacatcttggccttgcacctgtctccatcctccaaggacaactctgtatccagggccagctcaggggcaccctcaggtagctctgtcttctcgagaggctcagtcctctcgcctttgtcccaggacatctctaccaccagggcctcctgcagggatgcctcttcctggaagtccactgggttgatccactgccaagtctccttccctccagggaactcaatggcggagctggcacagggtgtgggcagcggcgggcacaggtgccgtgcggccctgttcaggccaaggtagccaaggacgcccacgagaaggatgctcaggaagctccccagggaggcgaagaagatgagccaatcagaaacctgcacttcgatgctgaagcgctggggctggctccagacacccctcagccacgctgtgtctgctcgcacctgcaccgtgtaggctacaccagcccgcaggccactgagggtaacttgggtctctgtgggctgcacgggatgctctgacacctgtttgctgtcttcatctcggcagcggacaacatactcctttaggacgccgggacaggtgctcagcagggatggtgcccagtccacagacacagagtccaagctatgattcttcaccgagacgtggtgcggtgtcccagctgctgaggcattgcccccaaagtggtaggtggacaggaccgtagaccacaaggtgagcttctcggggtgcgcagaggcaaagatggtaatgtagtaacacttttcctgccccattgccccagactctcgactccagctgtaggttgccattccagccggatccgggtcttgcggcgcagtcaggctgcaggtggcaaggcccccgtcctggcccacaggctgccattcaatgcaatacgtcatgctctgagcccgggctggccaatacatggtggtcccgttggttccgacgctgatattcagagccactggttctgtgtgggtgtcggcaggaatgtgccacgtctggttcaggccaggaccaaattggttcgaggagatgacagccacgttgtaggcagcacccgagagatagggcatcttccccaggtgcagggtcctggtggccttggccttacacgggcaggacagcatgtggagctgtagtcggtaagtgacctccgtgccaggcgccagcccttgacagccttctggaagctccagctgggttggctgctctttcagggtcagccgcctcctcccatcctggcccagctgctccaccgagaatctcacctgaggctgtggggggttttcagggggaacgcacacggggctgctccacttgctccaggaacttccttggctccccagctgccgtcgtcggagctggaattcctgggccacattcatctccagggggcagaggcaggactcagtatcatcatcctgaggtccgcagtcgcccaacttccatgggctgctgggtgtccggtgccggaactgcacctcagcaccaacctggttatccggggtctcccactccatacgcagctgcccggccaacttggacaccttgatgtctcccagaggaggctcatatttaactgagttgtagagctgcagggtcacctcaggagacttctctgtctggttcctggcccaggattccacccagagtgtgacagtgtacagcacagacaccccagcctggtcggagaactgcagcctggtggctgagccggcggcgaagtagcagcagcgcccggagctaaggcaacaccgcaggaagtggctgaccccagctgtgggaccctcatactgccaggagcactcgtaacgatcactggatatccgatagcatctcaggtccctagggcccgaggccgagcctgagtctgcatccggatatggcgggtcctgaaaacagcactcactggttctgcaggcagcgccctgcctggacagcaggaagaggaagaggagggggaccacccaggtcaccagcggctccatcggatccacgtagagccccacagccccaggggagcctctctgccacctgcgaggttcagccacc

SEQ ID NO: 35 - DNA, 1881 bp, Homo sapiens
ggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagatggcatgatctcagctcactgcaacctccgccttccagattcaagagattctcctgcttcagcctcccgagtagctgggattacaggcatctgccaccatacccggctaattttgtatttttagtagagacggggtttcaccacgttggccaggctggtctcgaactcctgacctcaagtgatccacctgccttggcctcccaaagtgttgggattataggcgtgagccaccatgcccagcctaatttttgtatttttagtagagatggagtttcaccatgttgcccaggctggtctcaaactcctgccctcaggtgatccacccacctcagcctctcaaagtgctgggattacaggtgtgagccactgtggccgacctactatttttattatttttgagctaggttctcagtctgttggcagactggagtgcaatcatggctcactgcagccttgaactcccagactcaagtgatccttccacctcagcctctggagtagctgggactacagacatgcaccaccacacctggttaattttttatttttattttttgtagagacaggtgtctctctacgttgcccaggctggtctcgaactcctgggctcaagtgatccacccatctccacctcccaaagtgctaggattacaggcgtgagccaccgtacccagcctggtcccatatcatagtgaaatggtgcctgtaaagctctcagcattggcttggcacatgcagttggtactcaataaacggctgttgctatccccaaaaaaaaaaaaaaaaaaaaaaaaa

SEQ ID NO: 36 - DNA, 1881 bp, Homo sapiens
tttttttttttttttttttttttttggggatagcaacagccgtttattgagtaccaactgcatgtgccaagccaatgctgagagctttacaggcaccatttcactatgatatgggaccaggctgggtacggtggctcacgcctgtaatcctagcactttgggaggtggagatgggtggatcacttgagcccaggagttcgagaccagcctgggcaacgtagagagacacctgtctctacaaaaaataaaaataaaaaattaaccaggtgtggtggtgcatgtctgtagtcccagctactccagaggctgaggtggaaggatcacttgagtctgggagttcaaggctgcagtgagccatgattgcactccagtctgccaacagactgagaacctagctcaaaaataataaaaatagtaggtcggccacagtggctcacacctgtaatcccagcactttgagaggctgaggtgggtggatcacctgagggcaggagtttgagaccagcctgggcaacatggtgaaactccatctctactaaaaatacaaaaattaggctgggcatggtggctcacgcctataatcccaacactttgggaggccaaggcaggtggatcacttgaggtcaggagttcgagaccagcctggccaacgtggtgaaaccccgtctctactaaaaatacaaaattagccgggtatggtggcagatgcctgtaatcccagctactcgggaggctgaagcaggagaatctcttgaatctggaaggcggaggttgcagtgagctgagatcatgccatctgtgtgggtgtcggcaggaatgtgccacgtctggttcaggccaggaccaaattggttcgaggagatgacagccacgttgtaggcagcacccgagagatagggcatcttccccaggtgcagggtcctggtggccttggccttacacgggcaggacagcatgtggagctgtagtcggtaagtgacctccgtgccaggcgccagcccttgacagccttctggaagctccagctgggttggctgctctttcagggtcagccgcctcctcccatcctggcccagctgctccaccgagaatctcacctgaggctgtggggggttttcagggggaacgcacacggggctgctccacttgctccaggaacttccttggctccccagctgccgtcgtcggagctggaattcctgggccacattcatctccagggggcagaggcaggactcagtatcatcatcctgaggtccgcagtcgcccaacttccatgggctgctgggtgtccggtgccggaactgcacctcagcaccaacctggttatccggggtctcccactccatacgcagctgcccggccaacttggacaccttgatgtctcccagaggaggctcatatttaactgagttgtagagctgcagggtcacctcaggagacttctctgtctggttcctggcccaggattccacccagagtgtgacagtgtacagcacagacaccccagcctggtcggagaactgcagcctggtggctgagccggcggcgaagtagcagcagcgcccggagctaaggcaacaccgcaggaagtggctgaccccagctgtgggaccctcatactgccaggagcactcgtaacgatcactggatatccgatagcatctcaggtccctagggcccgaggccgagcctgagtctgcatccggatatggcgggtcctgaaaacagcactcactggttctgcaggcagcgccctgcctggacagcaggaagaggaagaggagggggaccacccaggtcaccagcggctccatcggatccacgtagagccccacagccccaggggagcctctctgccacctgcgaggttcagccacc

SEQ ID NO: 37 - DNA, 4040 bp, Homo sapiens
tgcagagcacagagaaaggacatctgcgaggaaagttccctgatggctgtcaacaaagtgccacgtctctatggctgtgaacgctgagcacacgattttatcgcgcctatcatatcttggtgcataaacgcacctcacctcggtcaacccttgctccgtcttatgagacaggctttattatccgcattttatatgaggggaaactgacggtggagagagaattatcttgctcaaggcgacacagcagagcccacaggtggcagaatcccacccgagcccgcttcgacccgcggggtggaaaccacgggcgcccgcccggctgcgcttccagagctgaactgagaagcgagtcctctccgccctgcggccaccgcccagccccgacccccgccccggcccgatcctcactcgccgccagctccccgcgcccaccccggagttggtggcgcagaggcgggaggcggaggcgggagggcgggcgctggcaccgggaacgcccgagcgccggcagagagcgcggagagcgcgacacgtgcggcccagagcaccggggccacccggtccccgcaggcccgggaccgcgcccgctggcaggcgacacgtggaagaatacggagttctataccagagttgattgttgatggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtatcaatagtgatgaaattcaaatatgtggagcagagatcttcgttggtgttgctccagaacagcctcaaaatttatcctgcatacagaagggagaacaggggactgtggcctgcacctgggaaagaggacgagacacccacttatacactgagtatactctacagctaagtggaccaaaaaatttaacctggcagaagcaatgtaaagacatttattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttctgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctgagtggtgaggcttcaagccttaaagtcagtgtgccctcaaccagcacagcctgccccaattcccccagcccctgctccagcagctgtcatctctgggtgccaccatcggtctggctgcagctagaggacaggcaagccagctctgggggagtcttaggaactgggagttggtcttcactcagatgcctcatcttgcctttcccagggccttaaaattacatccttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagccttcaaaacaaatcaacctagttctatagatgaagacataaaagacactggtaaacaccaaggtaaaagggcccccaaggtggtcatgactggtctcatttgcagaagtctaagaatgtacctttttctggccgggcgtggtagctcatgcctgtaatcccagcactttgggaggctga

SEQ ID NO: 38 - DNA, 4040 bp, Homo sapiens
tcagcctcccaaagtgctgggattacaggcatgagctaccacgcccggccagaaaaaggtacattcttagacttctgcaaatgagaccagtcatgaccaccttgggggcccttttaccttggtgtttaccagtgtcttttatgtcttcatctatagaactaggttgatttgttttgaaggctctcaataatctaggagccatctagagctgtgcactccaatacagtagccactagccatgtgtggcgatttaaatttacaatcaaaatttaattcctcagtcaagctaaacacattccaagtgattaacagccaatgtgcctagtgtctaatgtactggacagtgagtgcagaggtagaacatttccatcatctcaggaagttctgttgaacagtactgatctgctgtgtacagcctgctgtgtagcaggtgctttagaaatagtcattgcatgaatggtcctctagactgtatgccattactactaagagctgtggtttctactctcctcctcatttccctttctggccagcataccaagaaagttactaggaattcaagttggagtctctaggtccacacagtgaaggatgtaattttaaggccctgggaaaggcaagatgaggcatctgagtgaagaccaactcccagttcctaagactcccccagagctggcttgcctgtcctctagctgcagccagaccgatggtggcacccagagatgacagctgctggagcaggggctgggggaattggggcaggctgtgctggttgagggcacactgactttaaggcttgaagcctcaccactcagagcatgagggagtcacacctcatctttaactgatccagagtcagcttatcaccacaggagaaggtgagtgggtgaagagaacttgaggggaaaacagaaagggagatgtgctgaggctccagttcttccagagagtcagcgagaggtgcctcatgtgaggggaggtcatctatgttggagggtaagtagccatcatgggtgggaaggtcacctgctgggagcaccgtccagggacaggctgggttttcgggctttgggtcggagcccctgctctccagcaccttgtacagatccaccagttgtctgctctcagcttggagagctcttggaggtggtgggcttgaggcactgtgcatcatgtctttctcagaggcctgatgaccttggattcctttttccctttgtggccagttggagcaggggggatgtctgaaaactggggtcacttgatgaaggacttcactgatgaccagcggttcaggatcttcaggcgtgggccagtctatcaggagcctgtccaagggcagctgtgtcttctcctctgcaatgggatatttcttagcgcaagtgctatttgctggatctggaatttctctgctacaccactgaggtctgagggctgctaggagaacaaacaccttttgctggaagtaatgcgttgagaaaatgcccaccatgatgatagcaatgcaaatgcttggtgccacaaacgccatccaattggctttaccttgcagacaaaattccctctcatttccgtgggaactttcaccagcagctgtcagagctgtcatccacaggacatatgtcactcggggctgcaggctgtttattggatgtgaattttgggagactctgtagggaatttcacagagctgaggctgggagttggagtcccgttccttccagtatatcctataatggaggaggcagcccatttgctcctggactggaatgctgttccatgaaattaaaatgctccccttttcctctgtgatggcattaatgtgggggccactcagtggtgctttgtgcttagagttacccaggatggagctgcatcctccttgatcccctgagagtgcatacacacggatttcataacagatgtaggattttatgttctctgaaatcagagcagacacattgtagggtcgactccgtagccagtttagagggacctgtgtgtcaccccctggatggagctctctccattccaccacgtactcctgaacagcagagggatctttcctgggaggctgccaagtcaccagaatgttgtccatgccctctgagtttgcagagacctggcgaggagccagcaaccctgcctcacacaggttcattatgttaatacgagtgggcagagaactgccttttgaatttgctgcagacacagccacagcccaatttccggttctaggaatgactgtggtccaggaggtgtgtcctgtgatgttctgtgtcatggctttccctcctgtcagctcctgcaaggtcacctgatagtggagaatttttcctcttgcctctgagacactcagattcttccagaaaagagaaatctgttgtctactgtagtcaatgtgccgtttcatgtaccagacatctaacatcccagtaggctcttcttctggtgtttgtgctctcaatgattcactccaatcactccaacttcccttataaagatgtagcttagaggaaatctgaaattcatattctgtaaatggtttcagatccagcaaatcatgtcttcctttggcctttgtaacattaaccatattccagagcctgctgttactgggccgatatctgagtcgattaagcagtaccagtccctcatctctccaataaagggtacatctgctcacagaagccttttgaaatttgattctaatgtcccacggaggaagaggcctcactatgtccaagaatgtgaatgtggatggaagtgaagaggagcttccaagactattgacagcagtaaccttggctgtgaaattggattcaggtgattcaggggtgaggttgattccaaagtccaaatagtcacaataaatgtctttacattgcttctgccaggttaaattttttggtccacttagctgtagagtatactcagtgtataagtgggtgtctcgtcctctttcccaggtgcaggccacagtcccctgttctcccttctgtatgcaggataaattttgaggctgttctggagcaacaccaacgaagatctctgctccacatatttgaatttcatcactattgatacaggccagtttgcagacaaacaaggttgtaccaaggggaagacctgtgacttgagaattgagggagtggccatggtgaaaattgattcttctgtcaaacttgtacaggattaacttgttacgtctggaatagtgaaagcagccttgtctgggcttcaaagagcatgtaatattgacagtggatccaagtaaaattacatgggaaggcttcacagtcacatcgcctctcttgcacgcatctatttttgctttaatcaacagccacgtgattataaacataaatgccaatgagcatcctctaaaagtatgtgccatcaacaatcaactctggtatagaactccgtattcttccacgtgtcgcctgccagcgggcgcggtcccgggcctgcggggaccgggtggccccggtgctctgggccgcacgtgtcgcgctctccgcgctctctgccggcgctcgggcgttcccggtgccagcgcccgccctcccgcctccgcctcccgcctctgcgccaccaactccggggtgggcgcggggagctggcggcgagtgaggatcgggccggggcgggggtcggggctgggcggtggccgcagggcggagaggactcgcttctcagttcagctctggaagcgcagccgggcgggcgcccgtggtttccaccccgcgggtcgaagcgggctcgggtgggattctgccacctgtgggctctgctgtgtcgccttgagcaagataattctctctccaccgtcagtttcccctcatataaaatgcggataataaagcctgtctcataagacggagcaagggttgaccgaggtgaggtgcgtttatgcaccaagatatgataggcgcgataaaatcgtgtgctcagcgttcacagccatagagacgtggcactttgttgacagccatcagggaactttcctcgcagatgtcctttctctgtgctctgca